(19) United States
(12) Patent Application Publication
(10) Pub. No.: US 2011/0004951 A1
(43) Pub. Date: Jan. 6, 2011

(54) METHODS AND COMPOSITIONS FOR IDENTIFYING COMPOUNDS THAT INHIBIT HIV-1 SUBUNIT-SPECIFIC REVERSE TRANSCRIPTASE

(75) Inventors: John C. Kappes, Birmingham, AL (US); Alok Mulky, Birmingham, AL (US); Xiaoyun Wu, Birmingham, AL (US)

Correspondence Address: McKeon Meunier Carlin & Curfman LLC, 817 W. Peachtree Street, Suite 900, Atlanta, GA, 30308, US

(73) Assignee: UAB Research Foundation, The

(21) Appl. No.: 10/599,851

(22) PCT Filed: May 24, 2005
(86) PCT No.: PCT/US05/18335

§ 371 Date: Jul. 30, 2009

Related U.S. Application Data
(60) Provisional application No. 60/573,918, filed on May 24, 2004.
(60) Provisional application No. 60/668,858, filed on Apr. 6, 2005.

(51) Int. Cl. A01K 67/00; C12Q 1/70; C07D 265/36; C07D 487/14; C12N 15/63; C12N 5/10; G01N 33/567; A61K 31/538; A61K 31/551; A61P 31/18; A61P 31/14

(52) U.S. Cl. 800/13; 435/5; 544/105; 540/578; 435/320.1; 435/325; 435/7.21; 514/230.5; 514/220

(57) ABSTRACT

This invention relates to methods and compositions for identifying compounds that inhibit HIV-1 subunit-specific reverse transcriptase.

### CROSS-REFERENCE TO RELATED APPLICATIONS

This application claims priority to U.S. provisional applications Ser. No. 60/573,918 filed on May 24, 2004 and Ser. No. 60/668,858, filed Apr. 6, 2005, which are herein incorporated by reference in their entirety

This invention was funded by the National Institutes of Health, Grant No. AI47714. Therefore, the United States Government may have certain rights in this invention.

### BACKGROUND OF THE INVENTION

### Background Art

The HIV type 1 (HIV-1) reverse transcriptase (RT) is required for the conversion of genomic RNA into double-stranded proviral DNA, catalyzed by the RNA-dependent DNA polymerase and ribonuclease H activities of the enzyme. HIV-1 RT is an asymmetric dimer formed by the association of p66 and p51 polypeptides, which are cleaved from a large Pr¹⁶⁰GagPol precursor by the viral protease during virion assembly. p51 contains identical N-terminal sequences as p66, but lacks the C-terminal ribonuclease H (RNase H) domain (di Marzo et al. Science 231, 1289-1291, 1986). The structure of HIV-1 RT has been elucidated by x-ray crystallography in a variety of configurations, including unliganded (Rodgers et al. Proc. Natl. Acad. Sci. USA 92, 1222-1226, 1995), complexed to nonnucleoside RT inhibitors (Ren, et al. Nat. Struct. Biol. 2, 293-302, 1995), or complexed with double-stranded DNA either with (Huang et al. Science 282, 1669-1675, 1998) or without deoxynucleotide triphosphate (Jacobo-Molinaet al. Proc. Natl. Acad. Sci. USA 90, 6320-6324, 1993; Kohlstaedt et al. Science 256, 1783-1790, 1992). Such analyses have shown that p66 can be divided structurally into the polymerase and RNase H domains, with the polymerase domain further divided into the fingers, palm, thumb and connections subdomains. Although p51 has the same polymerase domains as p66, the relative orientations of these individual domains differ markedly, resulting in p51 assuming a closed structure.

The RT heterodimer represents the biologically relevant form of the enzyme; the monomeric subunits have only low catalytic activity (Restle, et al. J. Biol. Chem. 265, 8986-8988, 1990). Structural analysis reveals three major contacts between p66 and p51, with most of the interaction surfaces being largely hydrophobic (Becerra et al Biochemistry 30, 11707-11719, 1991; Wang et al. Proc. Natl. Acad. Sci. USA 91, 7242-7246, 1994). The three contacts comprise an extensive dimer interface that includes the fingers subdomain of p51 with the palm of p66, the connection subdomains of both subunits, and the thumb subdomain of p51 with the RNase H domain of p66.

Several single amino acid substitutions in HIV-1 RT have been shown to inhibit heterodimer association (Ghosh et al. Biochemistry 35, 8553-8562 1996; Wohrl et al. J. Biol. Chem. 272, 17581-17587, 1997; Goel et al. Biochemistry 32, 13012-13018, 1993). These include the mutations L234A, G231A and W229A, all located in the primer grip region of the p66 subunit, and L289K in the thumb subdomain. These mutations are not located at the dimer interface and probably mediate their effects indirectly through conformational changes in the p66 subunit.

Several biochemical assays have been used previously to specifically measure RT dimerization. Some are based on the physical separation of monomers and dimers as determined by analytical ultracentrifugation and gel filtration. Other assays include intrinsic tryptophan fluorescence (Divita et al. FEBS Lett. 324, 153-158, 1993), chemical crosslinking (Debyseret al. Protein Sci. 5, 278-286, 1996), the use of affinity tags (Jacques et al J. Biol. Chem. 269, 1388-1393, 1994) and polymerase activity itself. Although these methods detect dimerization, they either lack specificity or are not easy to perform. Moreover, these assays do not facilitate the rapid genetic analysis of protein-protein interactions under physiological conditions nor are they suitable for high throughput screening for RT dimerization inhibitors.

Understanding the role of the individual RT subunits in RNA-dependent DNA synthesis has been the focus of several studies. These used in vitro biochemical methods to analyze the enzymatic activity of purified recombinant HIV-1 RT heterodimers wherein either the p51 or p66 subunit was selectively mutated (Boyer et al., 1994; Hostomsky et al., 1992; Le Grice et al., 1991). There remains a need in the art, however, for in vivo methods and compositions for identifying compounds that inhibit HIV-1 subunit-specific reverse transcriptase.

### SUMMARY OF THE INVENTION

In accordance with the purpose(s) of this invention, as embodied and broadly described herein, this invention, in one aspect, relates to a cell comprising a vector wherein the vector expresses a fusion protein comprising a p66 subunit, a p51 subunit, and Vpr, and a reverse transcriptase deficient proviral DNA.

In another aspect, the invention relates to a method of screening for a compound that inhibits viral reverse transcriptase.

This invention also relates to a method of screening for a compound that inhibits or enhances dimerization of a p66 subunit polypeptide of reverse transcriptase and a p51 subunit polypeptide of reverse transcriptase.

hi another aspect, this invention relates to a method of making a pharmaceutical composition and compounds identified by the methods described herein.

### BRIEF DESCRIPTION OF THE DRAWINGS

The accompanying drawings, which are incorporated in and constitute a part of this specification, illustrate embodiments of the invention and together with the description, serve to explain the principles of the invention.

FIG. 1 shows construction of the vpr-p51/p66 and FN expression plasmids. (A) Illustration of the vpr-p51/p66 expression plasmid. The vpr-p51/p66 expression plasmid was constructed to allow independent expression and subunit-specific analysis of p51 and p66. The vpr and p51 coding sequences were fused in-frame, while preserving the N-terminal protease cleavage (PC) site of RT by including 33 base-pairs of contiguous PR sequence 5′ of RT. A translational stop codon (TAA) was introduced to terminate RT expression at amino acids 440, which represents the full-length p51 subunit. vpr-p51 was succeeded by an internal ribosome entry site (IRES). The p66 coding region was inserted 3′ of the IRES and was modified to encode Met-Gly on the N-terminus. The vpr-p51/p66 expression plasmid was used to construct various p51/p66 mutants. Unless otherwise indicated, this was accomplished by inserting p51 or p66 DNA fragments at the BglII-MluI or XmaI-XhoI sites, respectively. (B) Illustration of the FN proviral construct. This proviral construct was made from the wild-type pSG3 plasmid using a previously described strategy (Dubay et al., 1992). The clone contains a 110 amino acid deletion (nucleotides 3374-3704) in the RT reading frame. Most of the RNase H domain and 13 amino acids of the carboxyl end of the polymerase domain were removed, leaving the IN coding region in-frame.

FIG. 2 shows a model for trans expression and packaging of heterodimeric RT. Cells are cotransfected with HIV-1 and the vpr-p51/p66 expression plasmids. Vpr-p51 incorporates p66 through interaction and stable association of the two subunits (Vpr-p51 and p66) within the cellular cytoplasm. Specific interaction between Vpr and Pr55^{Gag} leads to the incorporation of the Vpr-p51/p66 complex into progeny virions. Subsequent cleavage by the viral PR generates wild-type RT heterodimer (p51/p66).

FIG. 3 shows virion incorporation and proteolytic processing of trans-heterodimeric, RT. The FN proviral DNA was transfected alone or cotransfected with either the vpr-p66, vpr-p51/p66, or vprΔp51/p66 expression plasmids. Included as controls were the wild-type SG3 and the RT-IN minus SG3^{S-RT} proviruses. The transfection-derived virions were concentrated by ultracentrifugation, lysed and analyzed by immunoblotting using (A) anti-RT (α-RT), (B) anti-p66 (α-p66) or (C) anti-Gag (α-CA) antibodies.

FIG. 4 shows complementation of the M7 provirus eliminates non-Vpr-p51-mediated p66 incorporation. (A) Construction of the M7 proviral plasmid. The M7 construct was derived from the S-RT construct (Wu et al., 1997), which contains a TAA stop codon at the first amino acid positions of RT and IN. In addition to these mutations, M7 has a −1 frameshift at amino acid position 14 of RT, three stop codons, 441 (TAA), 444 (TGA) and 447 (TAG), in the RNase H domain and a RNase H catalytic site mutation at 443 (D443N). (B to D) Analysis of virion incorporation and proteolytic processing of Vpr-p51/p66. The M7 proviral DNA was transfected alone or together with the vpr-IN expression plasmid and either the vpr-p66, vpr-p51/p66, or vprΔp51/p66 expression plasmids. The wild-type SG3 was included as a control. Transfection-derived virions were concentrated by ultracentrifugation, lysed and analyzed by immunoblotting using (B) anti-RT (α-RT), (C) anti-p66 (α-p66) or (D) anti-Gag (α-CA) antibodies.

FIG. 5 shows infectivity of trans-heterodimeric complemented virions. Viruses were derived by transfection of 293T cells as described in FIG. 4 and analyzed for HIV-1 p24-ag concentration. Virus infectivity was analyzed using the TZM-b1 reporter cell line as described in Example 1. Infectivity is expressed as a percentage of the wild-type virus control. The results of three independent experiments are shown.

FIG. 6 shows subunit-specific analysis of the YMDD motif. The wild-type, control and mutated vpr-p51/expression plasmids, respectively, were cotransfected into 293T cells with the M7 and vpr-IN DNAs. Transfection derived viruses were analyzed for HIV-1′p24-ag concentration. (A) Analysis of infectivity. Infectivity was analyzed from three independent experiments. (B & C) Analysis of viral DNA synthesis. The DNA products of reverse transcription were analyzed as described in Example 1. (B) Early (R-U5) and (C) late (R-gag) products of reverse transcription were amplified from each DNA extract by PCR, resolved on 1.5% agarose gels and stained with ethidium bromide. To approximate the relative amount of each of the amplified DNA products, 10-fold serial dilutions of pSG3 DNA (ranging from 10¹ to 10⁵ copies) were prepared and analyzed in parallel. Distilled water (dw) was included as a negative control.

FIG. 7 shows interactions of the p51 YMDD (SEQ ID NO: 8) motif of HIV-1 reverse transcriptase at the junction of the p51 palm, p51 connection and p51 fingers subdomains in the structure of the RT/DNA/dNTP complex (pdb code 1RTD). The Trp-rich region is shown at the interface of the p51 and p66 subunits and proximal to the DNA-binding cleft.

FIG. 8 shows infectivity for Trp motif mutants. (A): Lane 1: trans-Vprp51/p66wild-type virus (15-20% of wild-type HIV-1). Normalized to 100%. Lane 2: Background control. Does not express p51 in the vpr-p51 reading-frame. Thus, it does not incorporate p66 via Vpr-p51 and there is no active RT in the virion (other than minimal amounts of p66 that could get non-specifically incorporated). Lane 3-9: Mutants in the tryptophan-repeat motif (Trp-motif) of RT. This motif is found the connection subdomain of RT and is unique in having 6 Trp residues. These residues form a hydrophobic cluster of 12 tryptophans spanning the dimerization interface between the RT subunits (p51 and p66). (B): Lane 1: trans-Vpr-p51/p66 wild-type virus. Lane 2: Background control. Does not express p51 in the vpr-p51 reading-frame. Thus, it does not incorporate p66 via Vpr-p51 and there is no active RT in the virion (other than minimal amounts of p66 that could get non-specifically incorporated). Lane 3-9: Mutants in the tryptophan-repeat motif (Trp-motif) of RT. The results of the RT assay are different in that clones like p51W401/p66 (lane 4) have background levels of activity (Vpr-Dp51/p66) in this biochemical assay (Example 2) although this mutant is wild-type on infectivity analysis.

FIG. 9 shows infectivity for p51W401-p66W410 dimer interface. (A) Lane 1: trans-Vpr-p51/p66 wild-type virus (15-20% of wild-type HIV-1). Normalized to 100%. Lane 2: Background control. Does not express p51 in the vpr-p51 reading-frame. Thus, it does not incorporate p66 via Vpr-p51 and there is no active RT in the virion (other than minimal amounts of p66 that could get non-specifically incorporated). The residues p51W401 and p66W410 are at the interface between p51 and p66 within interacting distance (˜3 Å) based on crystal structure. These residues were mutated both individually (lanes 3-6) and together (lanes 7-9). The single mutants do not have much effect on infectivity, while the double mutants have a greater effect. The p51/p66L234A (lane 10) and p51W401A/p66 (lane 11) are well-established dimerization defective mutants identified by biochemical and yeast-2-hybrid assay recognized in the field to be defective in RT assays (biochemical). It is quite clear from these controls that biochemical data do not accurately reflect what occurs in the virion. (B) Lane 1: trans-Vpr-p51/p66 wild-type virus (15-20% of wild-type HIV-1). Lane 2: Background control. Does not express p51 in the vpr-p51 reading-frame. Thus, it does not incorporate p66 via Vpr-p51 and there is no active RT in the virion (other than minimal amounts of p66 that could get non-specifically incorporated). The residues p51W401 and p66W410 are at the interface between p51 and p66 within interacting distance (˜3 Å) based on crystal structure. These residues were both mutated individually (lanes 3-6) and together (lanes 7-9). The single mutants to reduce RT (biochemical) activity to background levels (Vpr-Dp51/p66), while the double mutants have a greater effect and reduce the RT activity to negative control levels. The p51/p66L234A (lane 10) and p51W401A/p66 (lane 11), well-established dimerization defective mutants identified by biochemical and yeast-2-hybrid assay recognized in the field to be defective in RT assays (biochemical, Example 2) are also defective in our RT assay at negative control levels.

FIG. 10 shows structural analysis of RT connection subdomain. (A) Alignment of Trp-motifs of primate lentiviruses. The Pol amino acid sequences of representative strains of primate lentiviruses were aligned using MegAlign (DNASTAR, Inc.). HIV-1 RT sequence (amino acids 395-415) is shown along with corresponding alignments for other indicated primate lentiviruses. (B) Ribbon representation of the p66 and p51 subunits in the crystal structure of the complex of HIV-1 RT with double-stranded DNA and incoming tenofovir-diphosphate (pdb file 1T05) (Tuske et al. Nat. Struct. Mol. Biol. 11:469-74 (2004)). For clarity, only the protein is shown. The tryptophan-rich motif and other p51 residues at the interface of the two subunits are shown in Van der Waals volumes. Residues W401 and W410 of the p66 subunit are shown at or near the interface also in Van der Waals volumes. Residue W401 of the p66 subunit and residue N363 of the p51 subunit are shown at interacting distance at the subunit interface. (C) Magnification of the area in the box shown in “B”. Shown are the side-chains of residues of the tryptophan motif and of the interface that were mutated in this study. (D) Ribbon representation of the interface between p66 and p51. W410 of the p66 subunit is shown to have extensive interactions with residues of the p51 subunit (p51-N363, p51-W401, and p51-Y405).

FIG. 11 shows the analysis of p51 Trp-motif mutants. M7 proviral DNA was transfected into 293T cells alone or together with wildtype or mutant vpr-p51/p66 and vpr-lN expression piasmid DNAs. Transfection-derived vixions were analyzed by immunoblotting for (A) RT. (p51/p66) and (B) CA (p24). Expression of Vpr-p51 (C), p66 (D) and α-tubuiin (E) in the transfected 293T was examined by immunoblotting. (F) Infectivity of p51 Trp-motif mutants. The infectivity of virions containing alanine substitutions in the p51 Trp-motif was analyzed using the TZM-b1 reporter cell line as described in Example 4. Infectivity is expressed as a percentage of the wildtype trans-RT heterodimer (Vpr-p51/p66) complemented virions.

FIG. 12 shows the analysis of Trp-motif residues located at the RT heterodimer interface. Trp-motif residues that lie within interacting distance at the dimer interface were mutated. The infectivity of virions containing single (A) or dual (B) mutations was analyzed by the TZM-b1 reporter cell assay. Infectivity is expressed as a percentage of the wildtype trans heterodimer control.

FIG. 13 shows the analysis of W401 and W410 mutations in proviral DNA. (A) The importance of RT Trp-motif residues W401 and W410 for viral infectivity was analyzed using the HIV-1 NL4-3 molecular clone. Infectivity was determined using TZM-b1 reporter cells and the results are expressed as a percentage of wildtype NL4-3. Virions derived by transfection of the wildtype and mutant proviral DNAs were also analyzed by immunoblotting using mAbs to RT (B) and CA (C).

FIG. 14 shows subunit specific analysis of the W401A mutant. The W401 residue was mutated in p51, p66 or p51 and p66. Transfection derived virions containing the respective mutant trans TR's, were analyzed for (A) infectivity on TZM-b1 cells and (B) virion incorporation of p51 and p66 by immunoblotting. (C) Virion infectivity was determined using the TZM-b1 reporter cells (black bars) and the JLTRG-R5 reporter T cell line (white bars). Infectivity is expressed as a percentage of the wildtype trans-RT control.

FIG. 15 shows the effect of NNRTIs on RT subunit interactions. Virions were generated by cotransfection of 293T cells with M7 and trans-RT dimerization-defective mutant plasmid vpr-p51^{W401A}/p66^{W401A.} The dimerization enhancing NNRTI EFV was added to the culture medium 12 h after DNA transfection at concentrations ranging from 0.01-1.0 μM. The transfection-derived virions were collected 48 hours later and analyzed by immunoblot using mAbs to (A) RNase H and (B) CA.

FIG. 16 shows an analysis of infectivity. Transfection-derived viruses were analyzed for infectivity using the TZM-b1 reporter cell line as described in Example 1. Results are expressed as a percentage relative to an equal amount of wild-type SG3 virus.

FIG. 17 shows an analysis of complementation using RT-deficient M7 virus. Increasing DNA concentrations of vpr-p51/p66 or vpr-Δp51/p66 (ranging from 0.5. to 3.0 μg) were transfected into 293T cells along with a constant amount of M7 (6 μg) and vpr-IN (1 μg). (A) Virion incorporation of trans-RT subunits. Transfection-derived virions were concentrated by ultracentrifugation, lysed and analyzed by immunoblotting using anti-RT MAb (8C4). (B) Analysis of infectivity. Virions were analyzed for infectivity using the TZM-b1 reporter cell line. Results are expressed as a percentage of the wild-type SG3 virus.

FIG. 18 shows alternative approaches for trans-heterodimeric RT complementation. (A) M7 virions derived by cotransfection with vpr-p51/p66 and vpr-IN or vpr-p51/p66-IN were analyzed for infectivity by the TZM-b1 assay. Results are expressed as percentage compared to the wild-type SG3 virus. (B and C) Immunoblot analysis. The virions were examined for (B) p66 and (C) CA using MAbs specific for either the RNase H subdomain (7E5) or CA, respectively. (D) Infectivity analysis of virions generated by expressing two monocistronic RT constructs, vpr-p51 and LTR-p66. The + and − indicate the presence or absence of the plasmid included in the cotransfection, respectively. The amount of vpr-p51 used was kept constant (3 μg) while the LTR-p66 was transfected at increasing concentrations (1, 2 and 3 μg), indicated in parenthesis. M7 and vpr-IN were also included in the transfection. The transfection-derived virions were analyzed for infectivity using TZM-b1 cells.

FIG. 19 shows a distinction between Vpr-p51 and p66 by molecular mass. (A) Immunoblot analysis of virions derived by transfecting 293T cells with M7 and vpr-p51/p66 expression plasmids containing the different sized Pro-coding sequences. The 8C4 MAb was used as a probe to detect both the p51 and p66 subunits. (B) The transfection-derived virions were examined for viral infectivity using the TZM-b1 cells. Results are expressed as a percentage of the wild-type SG3 virus.

FIG. 20 shows inhibition of trans-RT using NRTI and NNRTI. Virions derived by cotransfection with M7, vpr-p51/p66 and vpr-IN were used to infect the TZM-b1 reporter cell line. The two RT drugs, 3TC and nevirapine, were analyzed at concentrations of 0.04-1.0 μM for 3TC and 1.0-25.0 μM for nevirapine as described in Example 1. The results are expressed as a percentage of untreated virus.

FIG. 21 shows enhancement of dimerization using Vpr-p51/p66. The RT heterodimerization enhancing drug, efavirenz (EFV), enhanced dimerization in a dose-dependent manner.

FIG. 22 shows that the 2′,5′-bis-O-(tert-butyldimethylsilyl)-beta-D-ribofuranosyl 3′-spiro-5″-(4″-amino-1″,2″-oxathiole 2″,2″-dioxide) (TSAO) exhibits inhibition characteristics similar to NNRTIs. The figure shows that TSAO can destabilize HIV-1 RT heterodimerization in p51 W401A/p66 W401A RT mutants.

FIG. 23 shows that there exists an interaction at the dimer interface that is important for subunit interaction. An analysis of wild type, L234A, W398A, W401A and YMAA mutations was conducted in the context of the complete HIV-1 NL4-3 proviral clone. The wildtype or mutant proviral DNAs were transfected into 293T ceils and progeny virions were analyzed for infectivity. The infectivity of virus containing a mutation was much less than that of wildtype, and was not rescued by efavirenz.

### DETAILED DESCRIPTION OF THE INVENTION

The present invention may be understood more readily by reference to the following detailed description of preferred embodiments of the invention and the Examples included therein and to the Figures and their previous and following description.

Definitions

As used in the specification and the appended claims, the singular forms “a,” “an” and “the” include plural referents unless the context clearly dictates otherwise. Thus, for example, reference to “a small molecule” includes mixtures of one or more small molecules, and the like.

Ranges may be expressed herein as from “about” one particular value, and/or to “about” another particular value. When such a range is expressed, this includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The terms “higher,” “increases,” “elevates,”, “enhances” or “elevation” refer to increases above basal levels, or as compared to a control. The terms “low,” “lower,” “reduces,” “inhibits” “decreases” or “reduction” refer to decreases below basal levels, or as compared to a control. For example, basal levels are normal in vivo levels prior to, or in the absence of, the vector, or addition of an agent such as or another small molecule or ligand.

The term “test compound” is defined, as any compound to be tested for its ability to interact with a selected cell.

The terms “control levels” or “control cells” are defined as the standard by which a change is measured, for example, the controls are not subjected to variables, but are instead subjected to a defined set of parameters in the absence of variables, or the controls are based on pre- or post-variable levels.

The terms “polypeptide,” “peptide,” and “protein” are used interchangeably throughout and are defined as sequences containing amino acids.

General

The biologically relevant, catalytically active form of human immunodeficiency virus type-1 (HIV-1) reverse transcriptase (RT) is a heterodimer consisting of a 51 kDa subunit and a 66 kDa subunit. Since p51 and p66 are derived from the same coding region, subunit-specific structure/function studies of RT have not been possible in vivo. RT has both DNA polymerase and RNase H activities that are required to convert the single-stranded RNA viral genome into double-stranded DNA upon entry of the virus into host cells.

RT is translated and assembled into virions as part of a larger Gag-Pol polyprotein precursor (Pr¹⁶⁰Gag-Pol). Proteolytic processing of Pr160^{Gag-Pol} by the pol-encoded protease (PR) generates the mature heterodimeric form (p51/p66) of the RT enzyme (Freed and Martin, 2001; Telesnitsky and Goff, 1997). The N-terminal 440 amino acids of p51 and p66 are collinear. The p66 subunit contains the DNA polymerase and RNase H domains, while the p51 subunit lacks the RNase H domain (Hizi et al., 1988; Larder et al., 1987a; Prasad and Goff, 1989). Elucidation of the HIV-1 RT structure has shown that the polymerase domain of p51 and p66 can be further divided into the fingers, palm, thumb, and connection subdomains (Kohlstaedt et al., 1992). Although both p51 and p66 contain each of these subdomains, their relative arrangements differ markedly between the two subunits. Since these subunits are derived from the same coding region, a mutation in the polymerase coding region generates a heterodimer that contains a mutation in each subunit. However, as their structures are different in the heterodimer, the effect of these mutations on RT subunit structure/function is not equivalent (Arnold et al., 1992; Kohlstaedt et al., 1992). Thus, the heterodimeric nature of RT has previously had limited detailed molecular genetic analyses of the p51 and p66 subunit function.

Viral and foreign proteins can be incorporated into virions by exploiting viral accessory proteins, such as HIV/SIV proteins Vpr or Vpx, as targeting vehicles. By expressing the desired protein in trans as a fusion with Vpr or Vpx, its incorporation is brought about through an interaction between Vpr/Vpx and the p6 domain of the cognate Gag precursor polyprotein (Lu et al., 1993; Paxton et al., 1993; Wu et al., 1994). Using this approach, it has been shown that HIV-1 RT and IN functions can be provided when expressed in trans as Vpr fusion proteins, independently of Pr160^{Gag-Pol} (Liu et al., 1997; Wu et al., 1999; Wu et al., 1997).

Herein described is a trans-complementation approach that enables the function of the individual RT subunits to be analyzed in the context of an infectious virus. For example, by cotransfecting cells with RT-defective proviral DNA and an LTR-vpr-p51-IRES-p66 expression cassette, it was demonstrated that Vpr-p51 interacts with p66 and mediates virion incorporation of a Vpr-p51/p66 heterodimeric complex. The p51 subunit was expressed as a Vpr-p51 fusion protein that incorporates into HIV-1 virions through an interaction between Vpr and the Gag precursor polyprotein. When coexpressed, p66 is specifically and selectively packaged as a Vpr-p51/p66 complex. Processing by the viral protease liberates Vpr and generates functional heterodimeric RT (p51/p66) that supports HIV-1 reverse transcription and virus infection (Example 1).

This approach was used to demonstrate that the YMDD aspartates of p66 are both required and sufficient for RT polymerase function, and that the p51 YMDD aspartates play a structural role that is required for viral cDNA synthesis in infected cells. By mutating D185 and D186 of either p51 or p66, the role of these residues, for the first time, in the context of an infectious virus, were studied. The results corroborate earlier findings that the aspartates of p66 (YMDD) are required and sufficient for polymerase function of the RT heterodimer. Decreased viral DNA synthesis and infectivity was observed with certain p51 aspartate mutations (YMDD), indicating that both the occupancy and charge of these residues are important for RT function in vivo. These findings demonstrate detailed molecular genetic and biologic analyses of the RT subunits in vivo.

Furthermore, disclosed herein is a subunit-specific mutagenesis approach that enables precise molecular analysis of the heterodimer in the context of infectious HIV-1 particles (Example 4). The contributions of amino acids comprising the Trp-motif to RT subunit interaction and function were analyzed. The results revealed important inter- and intra-subunit interactions of residues in the Trp motif. A tryptophan cluster in p51 (W398, W402, W406, W414), proximal to the interface, was found to be important for p51/p66 interaction and stability. At the dimer internee, residues W401, Y405 and N363 in p51 and W410 in p66 mediate inter-subunit interactions. The W401 residue is critical for RT dimerization (and therefore viral infectivity), exerting distinct effects in p51 and p66. The analysis of the RT heterodimerization enhancing non-nucleoside RT inhibitor (NNRTI), efavirenz, indicates that the effects of drugs on RT dimer stability can be examined in human cells. Thus, subunit-specific molecular interactions that affect RT heterodimer function and virus infection in vivo, have been elucidated Moreover, the ability to assess the effects of RT inhibitors on subunit interactions in a physiologically relevant context was demonstrated.

The first step in RT dimerization apparently involves interactions between hydrophobic residues in the connection subdomains of p51 and p66. This includes residues W401-W410 of p66 and residues P392-W401 of p51 (Rodriguez-Barrios et al. (2001); Morris et al. J. Biol. Chem. 274:24941-6 (1999); Tachedjian et al. J. Mol. Biol. 326:381-96 (2003)). The connection subdomain is distinctive in having six tryptophans and a tyrosine between amino acids 398-414. This motif is well conserved among the primate lentiviruses, and has been appropriately dubbed the tryptophan-repeat motif (Trp-motif). In a yeast two-hybrid approach to analyze Trp-motif mutations, residues p66^{W401} and p66W⁴¹⁴ were shown to be involved in RT dimerization (Tachedjian et al., 2003). Mutagenesis of other aromatic amino acids that lie between these two residues did not affect subunit interaction. Since p66^{W401} and p66^{W414} are not located at the dimmer interface, it appears that respositioning of structural elements between these residues accounted for their results.

Synthetic peptides corresponding to the connection subdomain (Trp-motif) have been reported to disrupt dimerization (Morris et al. (1999); Divita et al. (1994); Divita et al. J. Biol. Chem. 270:28642-6 (1995)). For example, a short peptide matching RT residues 395-404 was shown to inhibit heterodimerization in vitro and virus replication in cell culture (Morris et al. (1999)). Recent studies of nonnucleoside reverse transcriptase inhibitors (NNRTI) have heightened interest in compounds that interfere with RT conformational flexibility as a novel drug design concept (Sarafianos et al. Chem. Biol. 6:R137-46 (1999); Hughes et al. Proc. Natl. Acad Sci. USA 98:6991-2 (2001)). NNRTI are a group of small hydrophobic compounds with diverse structures that inhibit HIV-1 RT (see Balzarini et al. Curr. Top. Meal Chem. 4:921-44 (2004) for review). NNRTIs interact with HIV-1 RT by binding to a site on the p66 subunit of the heterodimer. This results in both short-range and long-range distortions of the RT structure. NNRTIs have been shown to interfere directly with the global hinge-bending mechanism that controls the cooperative motions of the p66 fingers and thumb subdomains required for RT function (Temiz et al. Proteins 49:61-70 (2002); Madrid et al. Proteins 45:176-82 (2001)). In yeast, several NNRTIs were shown to enhance p51/p66 subunit association as a result of a specific interaction of drug with p66 (Tachedjian et al. Proc. Natl. Acad. Sci. USA 98: 7188-93 (2001)).

The contribution of amino acid residues comprising the Trp-motif to RT subunit interaction and virus infection has been determined. Inter-subunit interactions between the connection subdomains include W401, Y405 and N363 in p51 and W410 in p66, and mutation of these residues impairs RT function and virus infectivity. The W401 residue of the Trp-motif was found to be of central importance. Mutation of this amino acid simultaneously in both subunits is deleterious to RT dimerization and virus infection. The RT heterodimerization enhancing drug, efavirenz (EFV), rescued this dimerization defect in a dose-dependent manner. Additionally, it was demonstrated that intra-subunit interactions between tryptophans comprising a hydrophobic cluster (W398, W402, W406, W414) proximal to the connection subdomain interface are important for p51/p66 subunit interaction and stability.

Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular plasmid is disclosed and discussed and a number of modifications that can be made to a number of molecules included in the plasmid are discussed, specifically contemplated is each and every combination and permutation of those molecules and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Disclosed herein are plasmids comprising a fusion protein comprising a p51-containing DNA fragment fused in frame and a viral accessory protein, such as vpr. By expressing the desired protein in trans as a fusion with Vpr, for example, its incorporation is brought about through an interaction between Vpr and the p6 domain of the cognate Gag, precursor polyprotein. The Vpr-p51 fusion includes the natural PR-RT cleavage site (PC), allowing processing by the viral protease and liberation of Vpr (Wu et al., 1997). Also disclosed herein is an expression cassette comprising LTR-vpr-p51-IRES-p66, wherein the nucleic acid comprises SEQ ID NO: 1.

Also disclosed herein are vectors comprising a p66 subunit, a p51 subunit, and Vpr, wherein Vpr and p51 are expressed as a fusion protein. The p66 and p51 subunits can be expressed on the same, or on different, mRNAs.

Optionally, an internal ribosome entry site (IRES) can be placed downstream of vpr-p51, followed by the p66 coding sequence. IRES are cis-acting RNA sequences able to mediate internal entry of a sequence on some eukaryotic and viral messenger RNAs upstream of a translation initiation codon. Examples of useful IRES can be found at http://ifr31w3.toulouse.inserm.fr/IRESdatabase, herein incorporated by reference in its entirety for the disclosure of various IRES.

Transcription of vpr-p51/p66 can then be placed under the control of a long terminal repeat (LTR), for example. LTRs are responsible for integration of the sequence into the host genome, initiation and enhancement of retroviral transcription, as well as transcriptional termination, and modulation of retroviral replication levels. Examples of LTRs useful with the plasmids described herein include SIV-LTR, HIV-1 LTR, and HIV-2 LTR, for example.

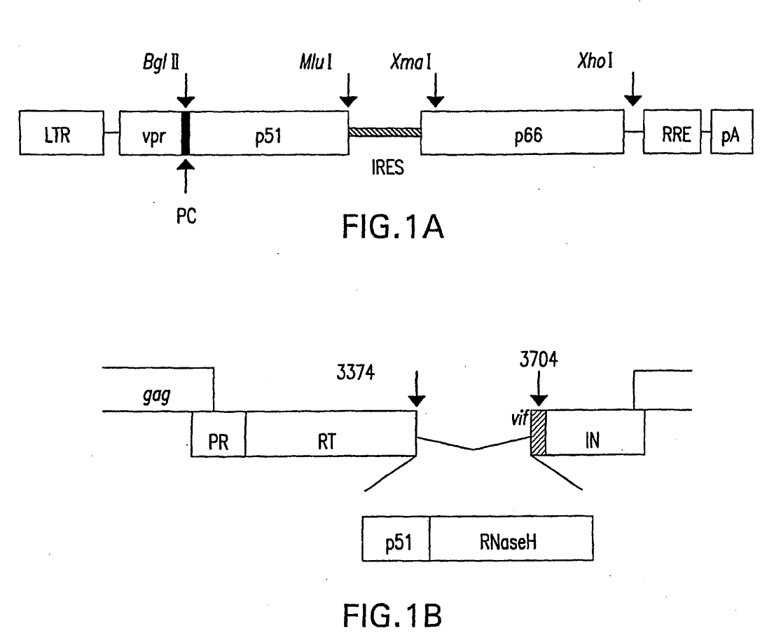

The plasmid can be incorporated into proviral clones that contain a deletion in RT. For example, the proviral clone pSG3^{FN} (FIG. 1B) was used to study incorporation of the heterodimeric trans-RT into virions when coexpressed with the vpr-p51/p66 expression plasmid (Example 1). The FN clone was selected for this purpose since it contains a deletion in RT that includes most of the RNase H region and extends 13 amino acids into the carboxyl-terminus of the p51 domain, however, any proviral clone can be used for this purpose. This created a defective RT, while the pol reading frame, including IN, remained open.

An expression plasmid including IN, such as vpr-IN, can also be included in conjunction with the plasmid disclosed herein. The M7 clone (pSG3^{FN}) does not express the IN protein, and integration of the nascent viral cDNA is required to detect infection. Moreover, IN is also required for efficient initiation of reverse transcription (Wu et al., 1999).

Effective trans-complementation requires expression of the two subunits (Vpr-p51 and p66), dimerization, and stable association of the p51 (Vpr-p51) and p66 subunits within the cytosol of the cell, specific interaction of Vpr with Pr55^{Gag}, incorporation of the Vpr-p51/p66 heterodimeric complex into virions, proteolytic cleavage to liberate Vpr from p51/p66, and proper interaction of RT with the template-primer.

Also disclosed herein are cells comprising: (i) a vector comprising a p66 subunit, a p51 subunit, and Vpr, wherein Vpr and p51 are expressed as a fusion protein; (ii) and a reverse transcriptase deficient proviral DNA. One example of a cell that can be used is the 293T cell.

Also disclosed are cell lines stably transformed with the plasmid described herein. For example, the cell line can comprise an exogenous nucleic acid, the nucleic acid comprising vpr-p51/66. The cell line can express viral nucleic acids as well, and can be induced to express viral nucleic acids by contacting the cell with a stimulus. An example of such a stimulus includes, but is not limited to, tetracycline. Also disclosed are transgenic animals expressing vpr-p51/66.

Homology/Identity

It is understood that one way to define any known variants and derivatives or those that might arise, of the disclosed genes and proteins herein is through defining the variants and derivatives in terms of homology to specific known sequences. For example SEQ ID NO: 1 sets forth a particular nucleic acid sequence encoding an expression protein and SEQ ID NO 2 sets forth a particular sequence of the protein encoded by vpr. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level. Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

Nucleic Acids

There are a variety of molecules disclosed herein that are nucleic acid based, including for example the nucleic acids of the plasmid disclosed herein, as well as those that encode the proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

Sequences

There are a variety of sequences related to, for example, the plasmid described herein, as well as any other protein disclosed herein that are disclosed on Genbank, and these sequences and others are herein incorporated by reference in their entireties as well as for individual subsequences contained therein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences.

Peptides

As discussed herein there are numerous variants of the vectors disclosed herein that are known and herein contemplated. In addition to the known functional variants there are derivatives of the proteins disclosed herein, such as Vpr, p51, or p66, which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis.

Specifically, mutations can occur in p51, p66, Vpr, IRES, or any of the nucleic acids encoding these peptides. Mutations can also occur in the env gene of HIV, for example, which can optionally affect the infectivity of the virus. These mutations can be deletions, substitutions, or insertion mutations. The mutations can occur in RT and/or in IN. The mutations can also be point mutations.

Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | AlaA |
| allosoleucine | AIle |
| arginine | ArgR |
| asparagine | AsnN |
| aspartic acid | AspD |
| cysteine | CysC |
| glutamic acid | GluE |
| glutamine | GlnK |
| glycine | GlyG |
| histidine | HisH |
| isolelucine | IleI |
| leucine | LeuL |
| lysine | LysK |
| phenylalanine | PheF |
| proline | ProP |
| pyroglutamic acid | Glu |
| Serine | SerS |
| Threonine | ThrT |
| Tyrosine | TyrY |
| Tryptophan | TrpW |
| Valine | ValV |

TABLE 2
Amino Acid Substitutions
iginal Residue Exemplary Conservative
Substitutions, others are known in the art.

Ala-ser
Arg-lys, gln
Asn-gln; his
Asp-glu
Cys-ser
Gln-asn, lys
Glu-asp
Gly-pro
His-asn; gln
Ile-leu; val
Leu-ile; val
Lys-arg; gln;
Met-Leu; ile
Phe-met; leu; tyr
Ser-thr
Thr-ser
Trp-tyr
Tyr-trp; phe
Val-ile; leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO: 1 sets forth a particular nucleic acid sequence of a vector described herein, which encodes the Vpr and p51 subunits; and SEQ ID NO: 2 sets forth a particular sequence of a Vpr protein. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Enginerring Reviews 13:197-216 (1995), Cahill et al., FIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO—(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Arm. Rev. Biochem. 61:387 (1992), incorporated herein by reference).

Functional Nucleic Acids

Functional nucleic acids are nucleic acid molecules that have a specific function, such as binding a target molecule or catalyzing a specific reaction. The compositions and methods described herein can be used with any functional nucleic acid. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of HIV or the genomic DNA of the subject, or they can interact with the polypeptide of the compositions disclosed herein. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following. U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

Methods

Also disclosed herein are methods of screening for a compound that inhibits viral reverse transcriptase comprising: a) contacting a cell comprising (i) a plasmid which expresses a fusion protein comprising a p66 subunit, a p51 subunit, and Vpr, (ii) and a reverse transcriptase deficient proviral DNA with the compound, and b) comparing the level of viral infectivity in the presence of the compound with the level of viral infectivity in the absence of the compound, wherein a decreased level of infectivity in the presence of the compound indicates that the compound inhibits reverse transcriptase.

The compositions and methods described herein can be used to treat retroviruses, and in particular lentiviruses. Lentiviruses share several molecular and pathogenic features that set them apart from other retroviruses. These include virus encoded regulatory proteins to stimulate viral gene expression, synthesis of multiply spliced mRNAs and chronic infection associated with slow development of disease. Lentiviruses include, but are not limited to, HIV-1, HIV-2 and SIV. In the methods described therein, the HIV or SIV particles can be derived by genes expressed in the cell, wherein the genes contain one or more nucleotide mutations. Examples of these specific mutations can be found in Example 1.

The p51 and p66 subunits can be expressed on the same or on different messenger RNAs. Furthermore, expression of Vpr-p51 can incorporate the p66 protein into viral particles. The plasmid can also express an internal ribosome entry site (IRES), as described above.

Also disclosed are methods of screening for a compound that inhibits dimerization of a p66 subunit polypeptide of reverse transcriptase and a p51 subunit polypeptide of reverse transcriptase comprising: a) contacting a cell comprising (i) a plasmid which expresses a fusion protein comprising a p66 subunit, a p51 subunit, and Vpr, (ii) and a reverse transcriptase deficient proviral DNA with the compound, and b) comparing the level of complex formation in the presence of the compound with the level of complex formation in the absence of the compound, a lower level of complex formation indicating that the compound inhibits dimerization of the p66 subunit and a p51 subunit.

Also disclosed are methods of screening for a compound that enhances dimerization of a p66 subunit polypeptide of reverse transcriptase and a p51 subunit polypeptide of reverse transcriptase comprising: a) contacting a cell comprising (i) a plasmid which expresses a fusion protein comprising a p66 subunit, a p51 subunit, and Vpr, (ii) and a reverse transcriptase deficient proviral DNA with the compound, and b) comparing the level of complex formation in the presence of the compound with the level of complex formation in the absence of the compound, a lower level of complex formation indicating that the compound enhances dimerization of the p66 subunit and a p51 subunit. Examples of compounds that inhibit reverse transcriptase by enhancing subunit dimerization include, but are not limited to, NNRTI.

Also disclosed is a method of inhibiting viral reverse transcriptase comprising contacting (1) the p51 subunit polypeptide, (2) the p66 subunit polypeptide, or (3) both the p51 subunit polypeptide and the p66 subunit polypeptide, with an effective amount of the compound identified by the method described above, thereby inhibiting viral reverse transcriptase.

Also disclosed is a method of inhibiting dimerization of a p51 subunit polypeptide of HIV-1 reverse transcriptase and a p66 subunit polypeptide of HIV-1 reverse transcriptase, which comprises contacting either (1) the p51 subunit polypeptide, (2) the p66 subunit polypeptide, or (3) both the p51 subunit polypeptide and the p66 subunit polypeptide, with an effective amount of the compound identified by the method described above, thereby inhibiting dimerization of the p51 subunit polypeptide of HIV-1 reverse transcriptase and a p66 subunit polypeptide of HIV-1 reverse transcriptase.

Also disclosed is a method of enhancing dimerization of a p51 subunit polypeptide of HIV-1 reverse transcriptase and a p66 subunit polypeptide of HIV-1 reverse transcriptase, which comprises contacting either (1) the p51 subunit polypeptide, (2) the p66 subunit polypeptide, or (3) both the p51 subunit polypeptide and the p66 subunit polypeptide, with an effective amount of the compound identified by the method described above, thereby enhancing dimerization of the p51 subunit polypeptide of HIV-1 reverse transcriptase and a p66 subunit polypeptide of HIV-1 reverse transcriptase.

In the methods described above, HIV-1 reverse transcriptase can be present in a subject, a eukaryotic cell, or a prokaryotic cell, for example.

The compounds disclosed herein can be NNRTIs. NNRTIs are a chemically diverse group of largely hydrophobic compounds that inhibit HIV-1 RT by binding in a hydrophobic pocket near the polymerase active site in the p66 subunit. NNRTIs have been described that can either stabilize or destabilize the RT heterodimer. Various NNRTIs have also been found to induce increased β-gal activity in the yeast two-hybrid system, due to enhanced RT subunit association 30. In particular, efavirenz binding to the NNRTI hydrophobic pocket enhanced RT heterodimerization, including RT with p51/p66 W401 mutations. Additionally, both the 2′,5′-bis-O-(tert-butyldimethylsilyl)-beta-D-ribofuranosyl 3′-spiro-5″-(4″-amino-1″,2″-oxathiole 2″,2″-dioxide) (TSAO) thymine derivatives and the N-acylhydrazones are classes of compounds that show inhibition characteristics similar to NNRTIs. Although these drugs may not bind to the well-defined NNRTI binding pocket of HIV-1 RT, they bind to a region of RT close to and partially overlapping this site. Furthermore, in the presence of a denaturant like urea these compounds have been shown to destabilize HIV-1 RT heterodimerization. Results show a dose-dependent enhancement of dimerization of the p51^{W401A}/p66^{W401A} RT mutant in the presence of efavirenz. (Example 4).

As used throughout, by a “subject” is meant an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. Preferably, the subject is a mammal such as a primate, and, more preferably, a human.

The methods of screening described herein are useful with high throughput screening methods. Screening optionally takes place in multi-well plates. Multi-well plates are standard in the art and come in a variety of sizes and shapes. For example, the multi-well plate can be 24, 48, or 96 well plates. Such screening assays can be automated or further modified for high throughput analysis. For high throughput screening, each well can include numerous test components. Ha positive reaction is detected in a well, the screening is repeated with one of the test compounds contained in a single well.

Optionally, reverse transcriptase containing (vpr-p51/p66) virus particles can be made that either lack Env or contain either autologous or heterologous Env derived by pseudotyping. Wei shows this with autologous Env, (Wei, X., J. M. Decker, H. Liu, Z. Zhang, R. B. Arani, J. M. Kilby, M. S. Saag, X. Wu, G. M. Shaw, and J. C. Kappes. 2002. Emergence of resistant human immunodeficiency virus type 1 in patients receiving fusion inhibitor (T-20) monotherapy. Antimicrob Agents Chemother 46:1896-905; Wei, X., J. M. Decker, S. Wang, H. Hui, J. C. Kappes, X. Wu, J. F. Salazar-Gonzalez, M. G. Salazar, J. M. Kilby, M. S. Saag, N. L. Komarova, M. A. Nowak, B. H. Hahn, P. D. Kwong, and G. M. Shaw. 2003. Antibody neutralization and escape by HIV-1. Nature 422:307-12; both herein incorporated in their entireties for the teaching of autologous Env); while Wu shows this with the VSV-G Env (Wu, X., J. K. Wakefield, H. Liu, H. Xiao, R. Kralovics, J. T. Prchal, and J. C. Kappes. 2000. Development of a novel trans-lentiviral vector that affords predictable safety. Mol Ther 2:47-55, herein incorporated by reference in its entirety for its teaching of VSV-G Env). The Wei citations describe how env minus or env mutant virus can be rendered infectious by providing an envelope glycoprotein in trans.

Compounds and Methods of Making

Also disclosed herein are methods of making a pharmaceutical composition which comprises: a) determining whether a compound inhibits reverse transcriptase by the methods described herein; and b) admixing the compound with a pharmaceutically acceptable carrier.

Also disclosed are compounds identified by the methods described herein, as well as compositions comprising the compounds identified by the methods described herein. Such compositions can also comprise a carrier. The compound can be capable of inhibiting HIV-1. Optionally, the compound can be a nonnucleoside reverse transcriptase inhibitor, or a nucleoside reverse transcriptase inhibitor. These compounds are known to those of ordinary skill in the art.

The compositions of the invention can be administered in vivo in a pharmaceutically acceptable carrier. By “pharmaceutically acceptable” is meant a material that is not biologically or otherwise undesirable. Thus, the material may be administered to a subject, without causing undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions identified by the methods disclosed herein can be administered orally, parenterally (e.g., intravenously), by intramuscular injection, intravenously, subcutaneously, intramuscularly, by intraperitoneal injection, transdermally, extracorporeally, topically or the like, including topical intranasal administration or administration by inhalant. topically or by liposome-mediated delivery. As used herein, “topical intranasal administration” means delivery of the compositions into the nose and nasal passages through one or both of the nares and can comprise delivery by a spraying mechanism or droplet mechanism, or through aerosolization of the small molecule or ligand. Administration of the compositions by inhalant can be through the nose or mouth via delivery by a spraying or droplet mechanism. Delivery can also be directly to any area of the respiratory system (e.g., lungs) via intubation.

The dosage of the compositions required will vary from subject to subject, depending on the species, age, weight and general condition of the subject, the severity of the infection being treated, the particular active agent used, its mode of administration and the like. Thus, it is not possible to specify an exact amount for every composition. However, an appropriate amount can be determined by one of ordinary skill in the art using only routine experimentation given the teachings herein.

The materials may be in solution, suspension (for example, incorporated into microparticles, liposomes, or cells). These may be targeted to a particular cell type via antibodies, receptors, or receptor ligands.

Suitable carriers and their formulations are described in Remington: The Science and Practice of Pharmacy (19th ed.) (ed. A.R. Gennaro, Mack Publishing Company, Easton, Pa. 1995.) Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8.5, and more preferably from about 7.8 to about 8.2. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The terms “effective amount” and “effective dosage” are used interchangeably. The term “effective amount” is defined as any amount necessary to produce a desired physiologic response. Effective amounts and schedules for administering the compositions may be determined empirically, and making such determinations is within the skill in the art. The dosage ranges for the administration of the compositions are those large enough to produce the desired effect in which the symptoms or disorder are affected. The dosage should not be so large as to cause substantial adverse side effects, such as unwanted cross-reactions, anaphylactic reactions, and the like. Generally, the dosage will vary with the age, condition, sex, type of disease and extent of the disease in the patient, route of administration, or whether other drugs are included in the regimen, and can be determined by one of skill in the art. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

Parenteral administration of a nucleic acid or vector to a subject is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. For additional discussion of suitable formulations and various routes of administration of therapeutic compounds, see, e.g., Remington: The Science and Practice of Pharmacy (19th ed.) ed. A.R. Gennaro, Mack Publishing Company, Easton, Pa. 1995.

Also, provided are kits for screening for compounds comprising a plasmid which expresses a fusion protein comprising a p66 subunit, a p51 subunit, and Vpr, and a reverse transcriptase deficient proviral DNA. Also provided are kits comprising a cell comprising the plasmid. Also provided are kits for treating viral infections comprising a composition identified by the methods disclosed herein.

The present invention is more particularly described in the following examples, which are intended as illustrative only since numerous modifications and variations therein will be apparent to those skilled in the art.

Although the present process has been described with reference to specific details of certain embodiments thereof, it is not intended that such details should be regarded as limitations upon the scope of the invention except as and to the extent that they are included in the accompanying claims.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., Amounts, temperature, etc.), but some errors and deviations should be accounted for. Unless indicated otherwise, parts are parts by weight, temperature is in ° C. or is at ambient temperature, and pressure is at or near atmospheric.

### Examples

### Example 1

### Subunit-Specific Analysis of the Human Immunodeficiency Virus Type-1 Reverse Transcriptase In Vivo

Expression and Virion Incorporation of Heterodimeric RT in Trans

For independent expression of the p51 and p66 subunits, the bicistronic pLR2P-vpr-p51-IRES-p66 (vpr-p51/p66) expression plasmid (abbreviations can be found in Table III) was constructed (FIG. 1A). The p51-containing DNA fragment was fused in frame with that of vpr. The Vpr-p51 fusion included the natural PR-RT cleavage site (PC), allowing processing by the viral protease and liberation of Vpr (Wu et al., 1997). The encephalomyocarditis virus internal ribosome entry site (IRES) was placed downstream of vpr-p51, followed by the p66 coding sequence. Transcription of vpr-p51/p66 was under control of the HIV-2 LTR (Wu et al., 1995).

TABLE III
Abbreviations for plasmids used in study

| Plasmid | Abbreviation |
|---|---|
| pSG3^{wt} | SG3 |
| pSG3^{S-RT} | S-RT |
| pSG3^{FN} | FN |
| pSG3^{M7} | M7 |
| pLR2P-vpr-p66 | vpr-p66 |
| pLR2P-vpr-Δp51-IRES-p66 | vpr-Δp51/p66 |
| pLR2P-vpr-p51-IRES-p66 | vpr-p51/p66 |
| pLR2P-vpr-p51-IRES- | vpr-p51/p66^{NN} |
| p66^{YMNN} | |
| pLR2P-vpr-p51^{YMNN}-IRES- | vpr-p51^{NN}/p66 |
| p66 | |
| pLR2P-vpr-p51^{YMAA}-IRES- | vpr-p51^{AA}/p66 |
| p66 | |
| pLR2P-vpr-p51^{YMEE}-IRES- | vpr-p51^{EE}/p66 |
| p66 | |
| pLR2P-vpr-p51^{YMKK}-IRES- | vpr-p51^{KK}/p66 |
| p66 | |
| pLR2P-vpr-IN | vpr-IN |

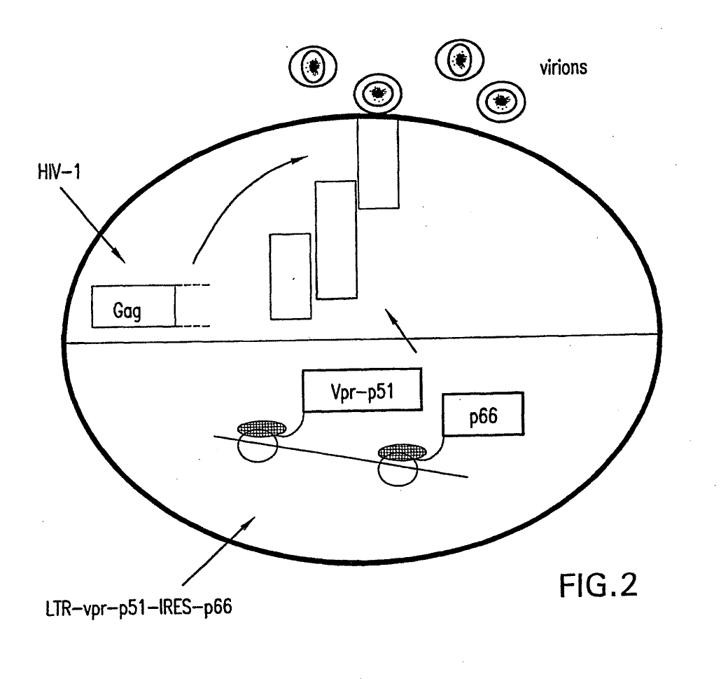

The proviral clone pSG3^{FN} (FN) (FIG. 1B) was used to study incorporation of the heterodimeric trans-RT into virions when coexpressed with the vpr-p51/p66 expression plasmid. The FN clone was selected for this purpose since it contains a deletion in RT that includes most of the RNase H region and extends 13 amino acids into the carboxyl-terminus of the p51 domain. This created a defective RT, while the pol reading frame, including IN, remained open. This overall strategy for studying subunit-specific RT function in the context of infectious virus is illustrated in FIG. 2. Effective trans-complementation would require expression of the two subunits (Vpr-p51 and p66), dimerization and stable association of the p51 (Vpr-p51) and p66 subunits within the cytosol of the cell, specific interaction of Vpr with Pr55^{Gag}, incorporation of the Vpr-p51/p66 heterodimeric complex into virions, proteolytic cleavage to liberate Vpr from p51/p66, and proper interaction of RT with the template-primer.

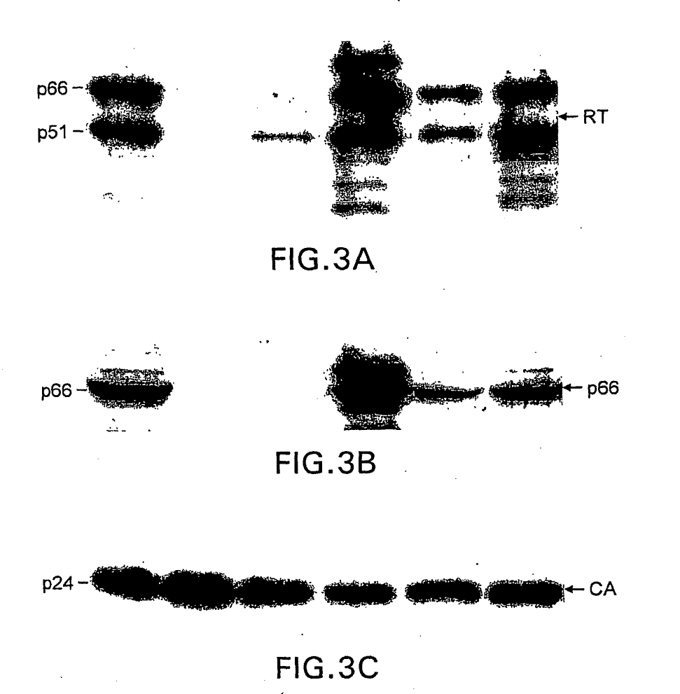

It was first determined whether the Vpr-p51 fusion protein could selectively incorporate p66 into virions. Virions derived by cotransfecting 293T cells with vpr-p51/p66 and FN were analyzed by immunoblot analysis. Using polyclonal anti-RT antiserum, two predominant proteins detected were consistent with the molecular masses of p51 and p66 (FIG. 3A, lane 6), and comigrated with those detected using SG3 virions (lane 1). Neither protein was detected using the RT-minus pSG3^{S-RT} (S-RT) virus (lane 2). Detection of the 51 kDa polypeptide with polyclonal anti-RT antibody showed that Vpr-p51 was packaged and processing by the viral protease liberated p51. The detection of the 66 kDa polypeptide showed incorporation of p66, however, the molecular mass of the unprocessed Vpr-p51 fusion protein is similar to that of p66. Therefore, a monoclonal antibody specific to the RNase H domain of p66 was used as a probe and confirmed incorporation of the trans-p66 subunit into virions (FIG. 3B, lane 6). As controls, virions produced by transfecting 293T cells with FN alone and FN in combination with the pLR2P-vprRT (vpr-p66) expression plasmid were analyzed. A protein comigrating with p51 that likely represents the truncated RT protein product (p51Δ13) was detected in virions generated by FN (lane 3). When the vpr-p66 expression plasmid was cotransfected with FN, p66, p51 and unprocessed Vpr-p66 were detected, in virions (lane 4). To determine whether the incorporation of p66 was mediated selectively by the Vpr-p51 fusion protein, virus derived by cotransfecting 293T cells with FN and pLR2P-vpr-Δp51-IRES-p66 (vpr-Δp51/p66) was analyzed (lane 5). The vpr-Δp51/p66 expression plasmid abrogates expression of the p51-coding region without affecting p66 expression. Using both polyclonal and monoclonal antibodies, a protein with a molecular mass equal to that of p66 was detected, showing that p66 incorporation, at least in part, was not selectively mediated by Vpr-p51 (lane 5). Immunoblot analysis using a monoclonal antibody against CA confirmed that approximately the same amount of each virus was analyzed (FIG. 3C).

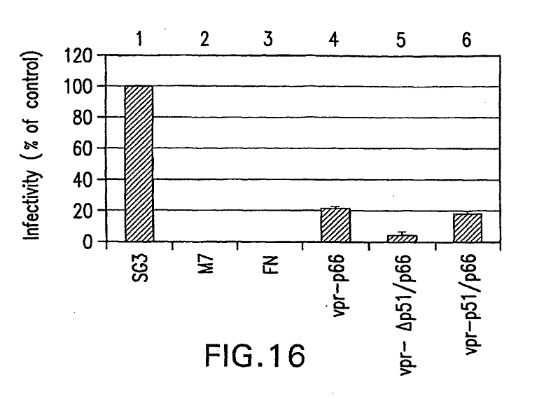

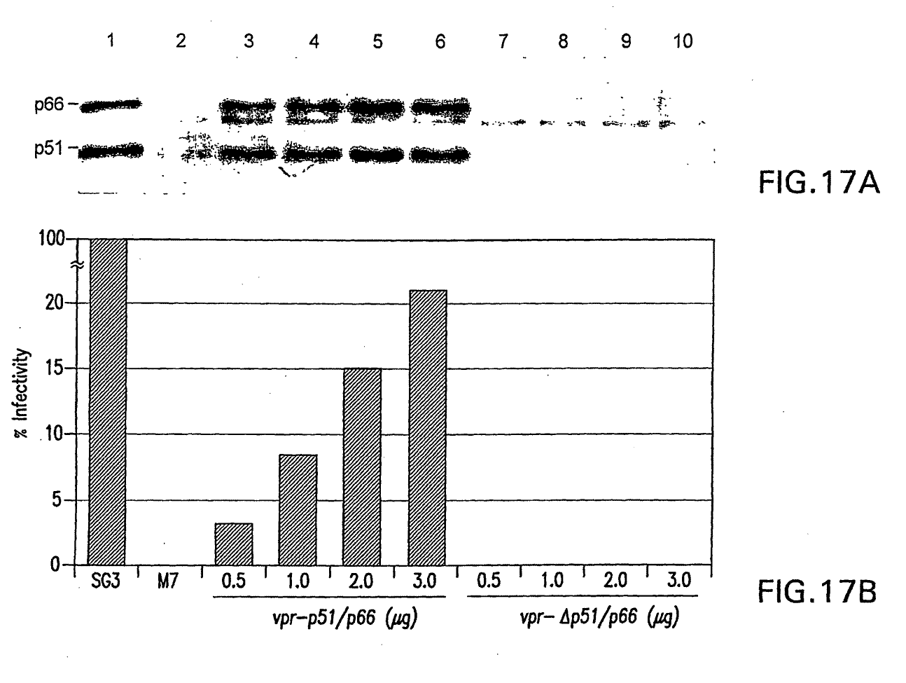

To examine whether the trans-heterodimeric RT could rescue the defect in FN infectivity, the transfection-derived virions were analyzed using the single cycle TZM-b1 reporter assay. In three independent experiments, cotransfection of the vpr-p51/p66 expression plasmid rescued FN infectivity to levels of 15-20% compared to wildtype SG3 virus (FIG. 16, lane 6). Virus derived by cotransfecting 293T cells with FN and vpr-p66 exhibited a similar level of infectivity (lane 4). The infectivity of FN virion derived by cotransfection with vpr-Δp51/p66 was approximately 3.5% of wildtype SG3 (lane 5). The RT-defective M7 and FN viruses had no detectable infectivity (lanes 2 and 3, respectively). These results showed that the heterodimeric trans-RT was to catalyze HIV-1 reverse transcription.

Specific Packaging of Heterodimeric RT

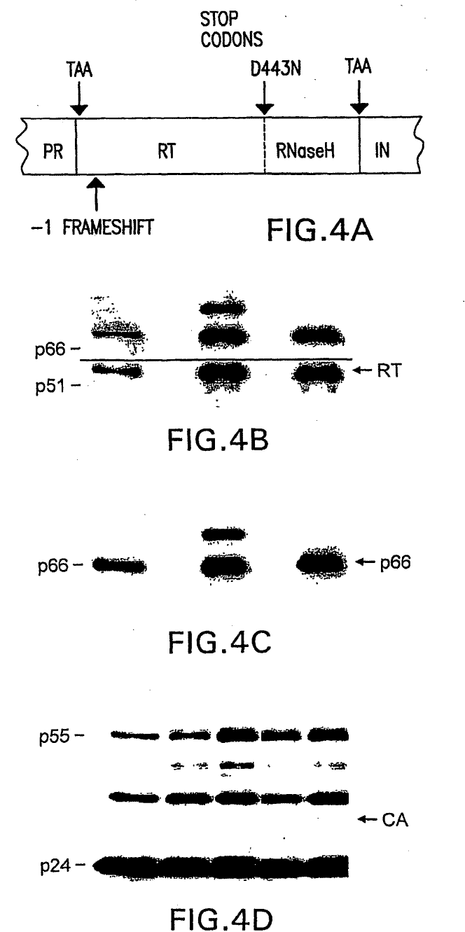

The strategy used for analyzing RT subunit function necessitates Vpr-p51-mediated selective incorporation of p66. Non-specifically packaged p66 can form p66/p66 homodimers and through proteolytic processing generate p51/p66 RT heterodimers, thus confounding the analysis of subunit-specific mutations. One explanation for the non-specific packaging of p66 observed in FIG. 3 was translational read-through of the TAA stop codon placed at the 5′ end of p51 in the vpr-Δp51/p66 expression plasmid. A second explanation was that the trans-p66 protein may associate intracellularly with the Gag-Pol polyprotein encoded by FN. Therefore, the pSG3^{M7} (M7) proviral clone was constructed. M7 has multiple mutations in the RT and IN coding regions (FIG. 4A) and was constructed to minimize the chance of encoding functional RT and IN, including that which conceivably could be generated via intermolecular genetic recombination with the vpr-p51/p66 plasmid. Virus generated by cotransfection of M7 with vpr-p51/p66 contained the p51 and p66 proteins, detectable with monoclonal anti-RT antibody (FIG. 4B, lane 5). In contrast to virus generated by FN (FIG. 3), virus generated by cotransfecting 293T cells with M7 and vpr-Δp51/p66 did not contain detectable p66 (lane 4). Probing blots with p66 monoclonal antibody confirmed selective, Vpr-p51-mediated packaging of p66 (FIG. 4C, lane 5). By probing a replica blot with monoclonal antibody against CA, it was confirmed that approximately the same amount of each virus was analyzed, and that the M7 virus did not have detectable abnormalities in either virion assembly or maturation (FIG. 4D). These results demonstrated that the Vpr-p51 fusion protein can selectively incorporate the p66 RT subunit into HIV-1 virions. Moreover, they indicate that the p51/p66 heterodimer is relatively stable, subsequent to virion incorporation. If not, free p66 might be expected to form homodimers that would be processed by viral PR, resulting in excess p51. However, FIG. 4B shows that a similar amount of each subunit was present in the M7 virions.

Heterodimeric Trans-RT Rescues the Infectivity of RT-Deficient Virus

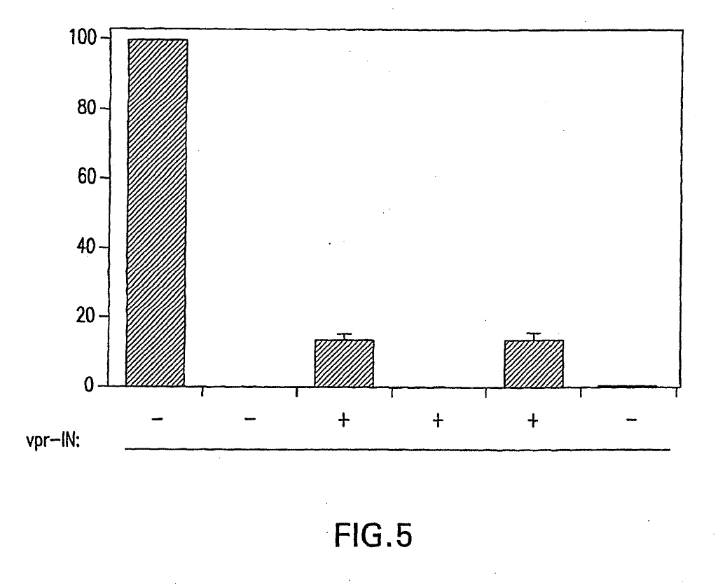

To determine if the heterodimeric trans-RT was functional, the M7 proviral construct was cotransfected into 293T cells with vpr-p51/p66 and vpr-IN. The vpr-IN expression plasmid was included since the M7 clone does not express the IN protein and integration of the nascent viral cDNA is required to detect infection using the TZM-b1 reporter cell line. Moreover, IN is also required for efficient initiation of reverse transcription (Wu et al., 1999). In three independent experiments, virus infectivity was rescued to about 15% of that of wild-type virus (FIG. 5, lane 5). Virus derived by cotransfecting 293T cells with M7, vpr-p66 and vpr-IN exhibited a similar level of infectivity (lane 3), consistent with earlier reports (Wu et al., 1997). The infectivity of M7 virus derived by cotransfection with vpr-Δp51/p66 and vpr-IN was less than 0.05% of wild-type virus (lane 4), or 0.2% compared with virus complemented with vpr-p51/p66. These results demonstrated that the heterodimeric trans-RT is functional, and with the M7 proviral clone, minimal complementation of virus infectivity was due to non-Vpr-p51 mediated packaging of p66. Furthermore, virus infectivity was not efficiently complemented without the IN protein (lane 6).

Subunit-Specific Analysis of the YMDD Motif

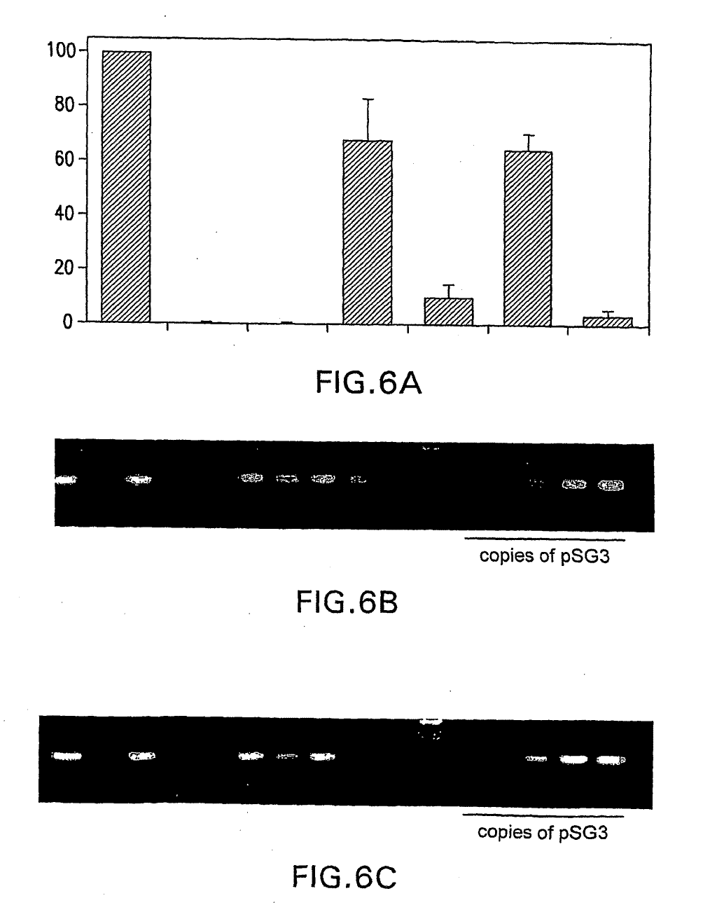

There exists a preponderance of evidence from biochemical and structural studies that shows HIV-1 reverse transcription is catalyzed by the p66 subunit of RT. However, the function of D185 and D186 in the p51 and p66 subunits, respectively, has not been directly tested in the context of an infectious virus. To study the function of these aspartate residues in one subunit independently of the other, either the p66 or the p51 coding region of the vpr-p51/p66 plasmid was mutated in both aspartates of the YMDD motif. Virus was analyzed for infectivity using the TZM-b1 reporter cell line and for DNA synthesis following acute infection of JC53 cells. Virus containing the p51/p66^{YMNN} mutant RT with Asp185Asn and Asp186Asn mutations in p66 was severely defective in infectivity (FIG. 6A, lane 3). Analysis of infected cells for viral DNA revealed a severe defect in reverse transcription (FIGS. 6B and C, lanes 5). The severity of this defect, showed that the p51 subunit of the heterodimer does not catalyze viral DNA synthesis in vivo. Moreover, when the equivalent catalytic site mutation was analyzed in p51 (p51^{YMNN}/p66), virus infectivity was reduced to approximately 70% of that of p51/p66 (wild-type) complemented virus (FIG. 6A, lane 4). Similarly, viral DNA synthesis of virus containing the p51^{YMNN}/p66 RT was also modestly reduced compared to that of wild-type (FIGS. 6B and C, lanes 6). This showed that the YMDD aspartates of p51 affect viral DNA synthesis.

To further analyze the role of these p51 aspartates, they were mutated to alanines, glutamates, or lysines. Virus stocks containing each mutant RT were prepared by cotransfection and analyzed for infectivity and DNA synthesis. Similar to the asparagine mutations, the glutamic acid mutations (p51^{YMEE}/p66) decreased virus infectivity and DNA synthesis only slightly (FIG. 6). More dramatic decreases in both DNA synthesis and virus infectivity were observed for viruses containing either the alanine (p51^{YMAA}/p66) or the lysine (p51^{YMKK}/p66) p51 mutations. The effect of each of the p51 YMDD mutants on viral DNA synthesis was examined using primer pairs that detect either early (R-U5) or late (R-gag) products of reverse transcription. The magnitude of the defect was similar with both primer pairs, showing that the defect was at or prior to initiation. The cellular expression of Vpr-p51 and p66 by these trans Vpr-RT constructs was equivalent, ruling out expression defects as the cause for differences in viral infectivity and DNA synthesis. The effect of each of these mutations on DNA synthesis and infectivity correlate with the disruptiveness of the mutation introduced. This shows that the YMDD motif of p51, specifically its aspartate residues, is important to maintain the structure of the RT heterodimer and its enzymatic function in vivo.

Effect of Expressing p66 and IN in cis

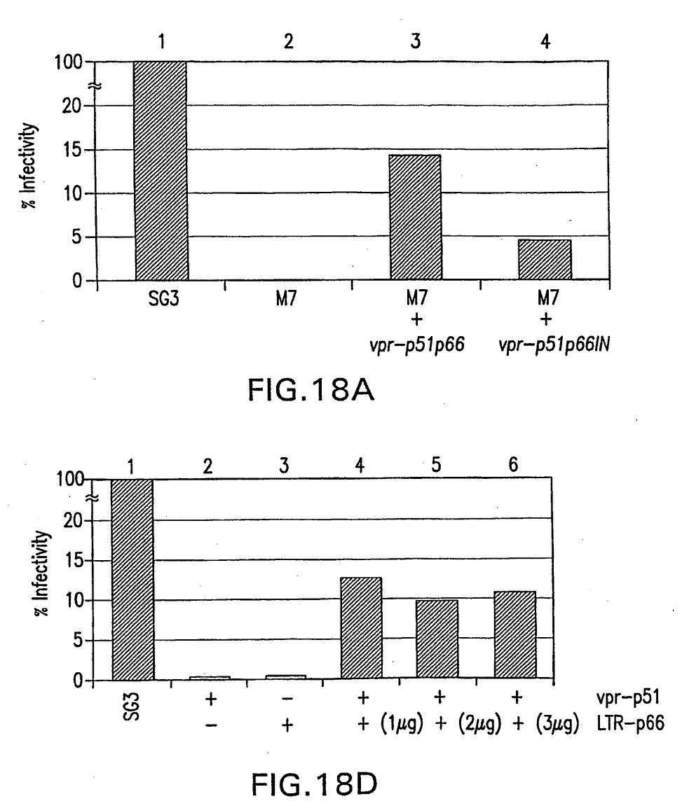

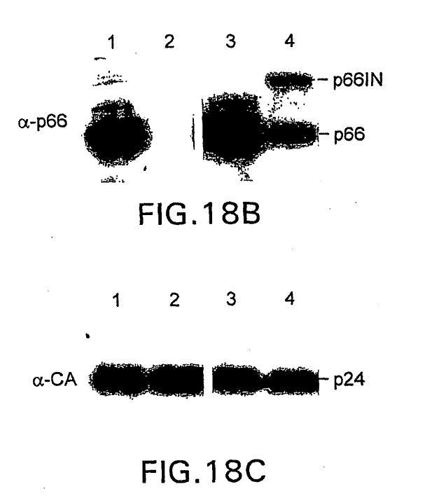

The results indicate the rescue of M7 infectivity to a maximal level of approximately 15% compared to wildtype SG3. This can be explained, at least in part, by reports showing defects in virions lacking RT-IN expression and packaging as a contiguous protein, included aberrant morphology and RNA conformation. In an attempt to enhance the complementation efficiency of the assay, a vpr-p51/p66-IN expression plasmid was constructed and cotransfected into 293T cells with M7. Progeny virions exhibited decreased infectivity (lane 4) compared to virions generated with vpr-p51/p66. Virions concentrated by ultracentrifugation were analyzed by immunoblotting using the p66 (RNase H) specific MAb. The vpr-p51/p66 complemented virions (FIG. 18B, lane 3) incorporated p66 at levels comparable to wildtype SG3 (lane 1), while the RT-minus M7 virions showed no p66 (lane 2). Virions generated by cotransfection of M7 and vpr-p51/p66-IN (lane 4) had reduced p66 compared to vpr-p51/p66-derived virions (lane 3), and also showed a relatively large amount of unprocessed p66-IN (RT-IN). Probing a replica blot with MAb to CA confirmed that approximately the same amount of each virus was analyzed (FIG. 18C).

Effect of Expressing p66 and vpr-p51 from Separated Plasmids

The efficiency of complementation when the Vpr-p51 and p66 subunits were expressed from separate mRNA in the transfected cells was examined. 293T cells were cotransfected with M7, pLR2P-vpr-p51 (vpr-51), pLR2P-p66 (p66) and vpr-IN and progeny virions were analyzed. Infectivity was rescued to levels similar to that exhibited previously using vpr-p51/p66, about 10-13% of wildtype SG3. Complementation analysis using either vpr-p51 or p66 only rescued infectivity by 0.1% and 0.2% of wildtype SG3, respectively (lanes 2 and 3). These results indicate rescue of M7 virion infectivity when p66 and Vpr-p51 are coexpressed from separate mRNAs. Expression of Vpr-p51 and p66 from separate genetic elements facilitates the manipulation of this approach for analyzing RT function, since this allows the ratios of the two plasmids to be varied.

Distinction between Vpr-p51 and p66 on Immunoblots

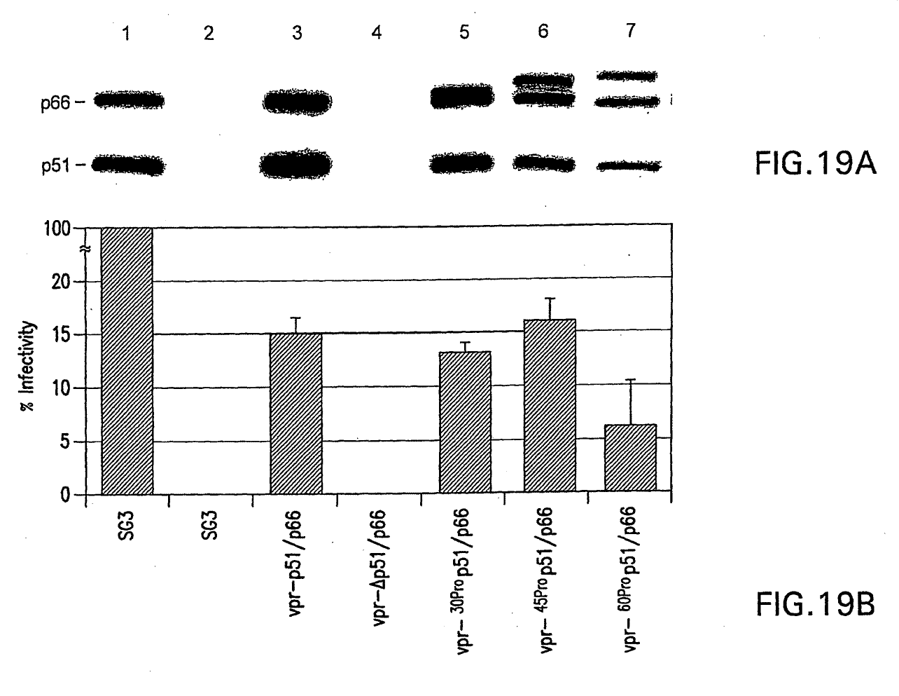

The vpr-p51/p66 construct places vpr and RT in-frame, and preserves the N-terminal protease cleavage site of RT by including 11 amino acids of PR (11Pro) between Vpr and RT. Thus, the molecular mass of the unprocessed Vpr-p51 fusion protein is indistinguishable from that of p66 when analyzed by Western blotting. This has necessitated the use of a MAb specific to the RNase H domain for specific detection of p66 in virions. To distinguish between these two proteins (Vpr-p51 and p66) by molecular mass, PR sequence encoding 30, 45 or 60 amino acids was introduced between the Vpr and p51 coding regions in vpr-p51 (vpr-^{30Pro}p51/p66, vpr-^{45Pro}p51/p66 and vpr-^{60Pro}p51/p66, respectively). Immunoblot analysis of virions derived by cotransfection of vpr-^{30Pro}p51/p66 along with M7 into 293T cells showed that adding 30 amino acids of PR was not sufficient to clearly differentiate Vpr-p51 and p66 (FIG. 19, lane 5). However, the addition of 45 or 60 PR residues allowed clear distinction between Vpr-p51 and p66 (lanes 6 and 7). Analysis of infectivity for the 30Pro and 45Pro-derived trans-RT containing virions indicated that they rescued M7 infectivity at levels comparable to the original (11Pro containing) vpr-p51/p66. In contrast, the 60Pro rescued infectivity less efficiently (lane 7). Taken together, these results indicate that vpr-^{45Pro}p51/p66 is a viable alternative to the original vpr-p51/p66 construct for analyzing trans-RT heterodimer structure/function. Specific detection of Vpr-p51 and p66 based on molecular mass can facilitate quantitative analyses of the heterodimer.

Chemotherapeutic Inhibition of the Trans-RT Heterodimer

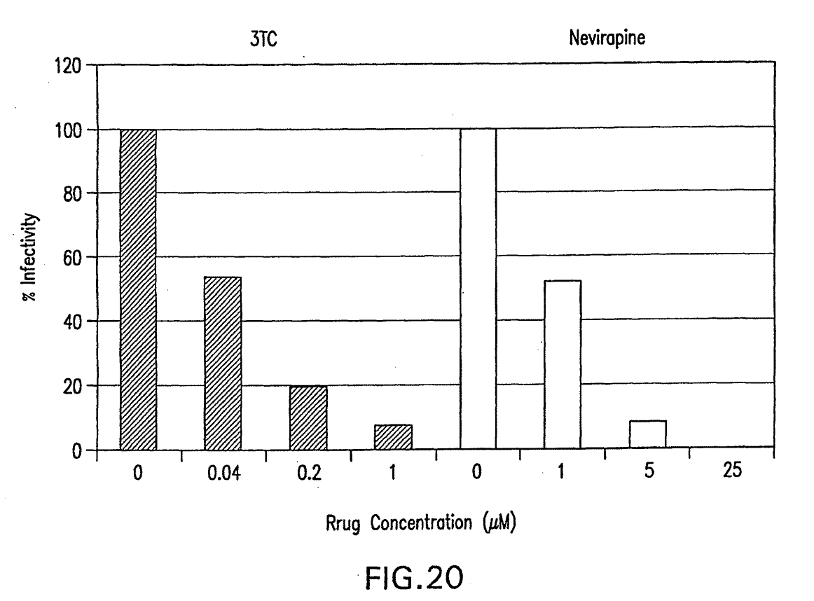

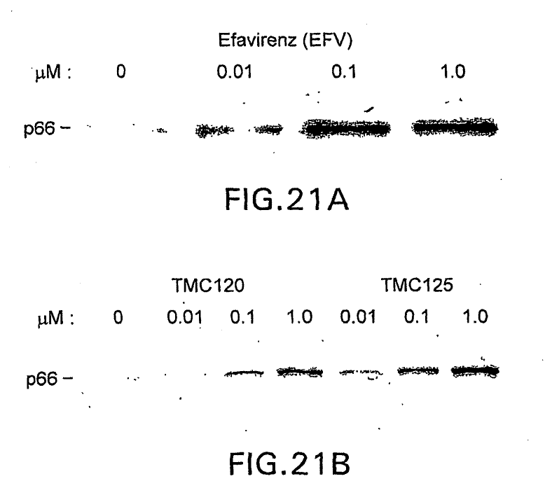

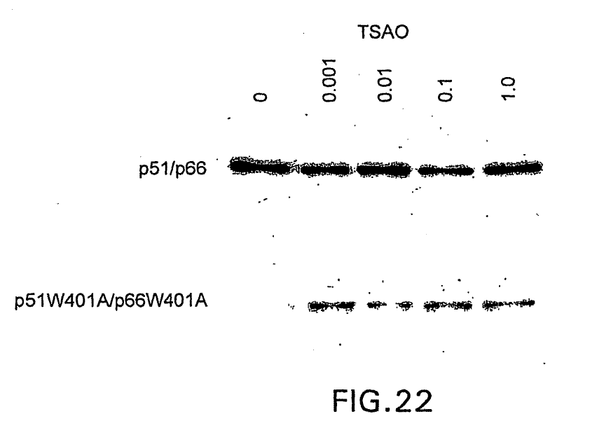

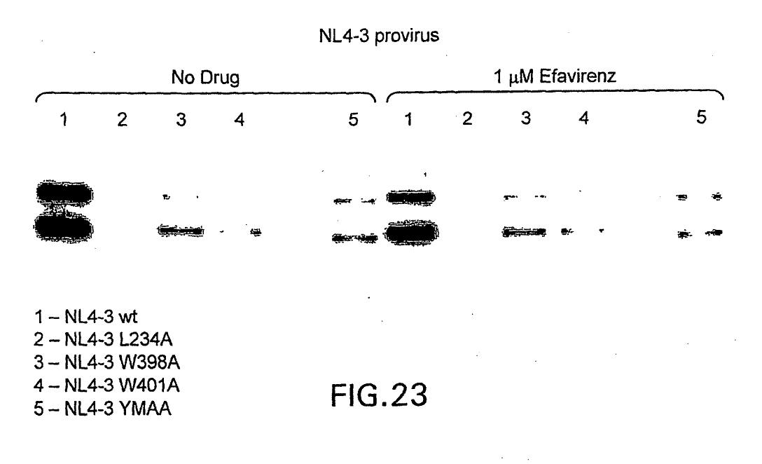

The trans-heterodimer assay is of clinical relevance for analyzing HIV-1 RT inhibitors, drug resistance and the effects of drug resistance mutations on viral fitness. To examine the response of the trans-heterodimeric RT to anti-RT drugs, transfection-derived M7 virions complemented with wildtype trans-RT (Vpr-p51/p66) were used to infect the TZM-b1 indicator cells in the absence or presence of either 3TC (0.04, 0.2 and 1.0 μM) or nevirapine (1.0, 5.0 and 5.0 μM). Both drugs exerted a potent, dosage-dependent antiviral effect, as evidenced by an inhibition of infectivity. The IC50s for 3TC and nevirapine were 0.138 and 0.011 mM, respectively. These results indicate that the effects of both NRTIs and NNRTIs on the trans-heterodimeric RT are similar to those observed for RT derived from the Gag-Pol precursor of HIV-1 provirus (FIG. 20).

Discussion

Vpr-p51 and p66 form an intracellular dimer (Vpr-p51/p66) that is specifically incorporated into virions, processed by the viral PR to liberate p51/p66, and rescues the infectivity of RT-deficient HIV-1. By analyzing mutations in the YMDD aspartates of either p51 or p66 the function of these residues in the context of an infectious virus was delineated. The absence of minus-strand strong-stop DNA synthesis in cells infected with virus, in which the YMDD aspartates of p66 were mutated, corroborates findings from previous in vitro studies, and demonstrates that in a heterodimer, p66 is solely responsible for the catalytic/polymerase function of RT in vivo. The analysis of the p51 subunit indicates that mutations in the YMDD aspartates impair virus infection and DNA synthesis due to an effect on RT structure rather than catalytic function.

The YXDD motif (SEQ ID NO: 9) of retroviral RTs is highly conserved and it has been described in the active site of many viral and cellular polymerases (Kamer and Argos, 1984; Toh et al., 1983). The HIV-1 YMDD motif is situated in the palm domain (Ding et al., 1998; Kohlstaedt et al., 1992; Sarafianos et al., 2001). The Y183 and M184 amino acid residues contribute to the dNTP binding pocket of p66 (Huang et al., 1998). While some substitutions of these residues are tolerated, most mutations at these sites reduce polymerase function (Lowe et al., 1991; Wakefield et al., 1992). The most conserved feature of the YMDD motif is the aspartates (D185 and D186), which together with a third aspartate (D110) form the polymerase catalytic triad. Mutation of the aspartates abolishes RT catalytic function and virus infectivity (Boyer et al., 1992; Larder et al., 1987b; Lowe et al., 1991). The role of the catalytic aspartates in each RT subunit has been studied by expressing p51 and p66 separately in Escherichia coli (Hostomsky et al., 1992; Le Grice et al., 1991). Recombinant heterodimers containing polymerase active site mutations exclusive to p51 retain almost wild-type levels of polymerase activity, whereas heterodimers containing the same mutation(s) in p66 appear to be defective in polymerase activity. In the RT heterodimer, the polymerase domain of p51 assumes a closed conformation (Kohlstaedt et al., 1992), and therefore p51 does not appear to play a catalytic role in reverse transcription in vivo.

The role of the p51 YMDD aspartates in reverse transcription was investigated by analyzing the effects that different mutations had on reverse transcription and virus infectivity. The p51 YMDD aspartates were substituted with both conservative and non-conservative amino acid residues. The YMNN mutant is relatively conservative, since asparagine is almost isosteric to, but less charged than, Asp. In the YMEE mutant the length of the side chain is increased by one methylene group without changing the negative charge. Similar to aspartate, asparagine and glutamine are capable of participating in hydrogen bond interactions through their side chains. These two p51 mutants caused a slight reduction of infectivity and DNA synthesis. Substitution of the p51 YMDD aspartates with either alanines (YMAA) or lysines (YMKK) drastically reduced infectivity and DNA synthesis. The alanines have a short hydrophobic side chain that cannot make hydrogen bonds with neighboring polar residues. The lysines present an opposite polarity through a lengthened side chain. Small changes in charge and/or length of the side-chain can be tolerated (i.e. YMNN and YMEE), however, a charge shift and/or substantial changes in side chain length are not (i.e. YMKK and YMAA). These findings show that side chain interactions of p51 YMDD aspartates are important for RT function.

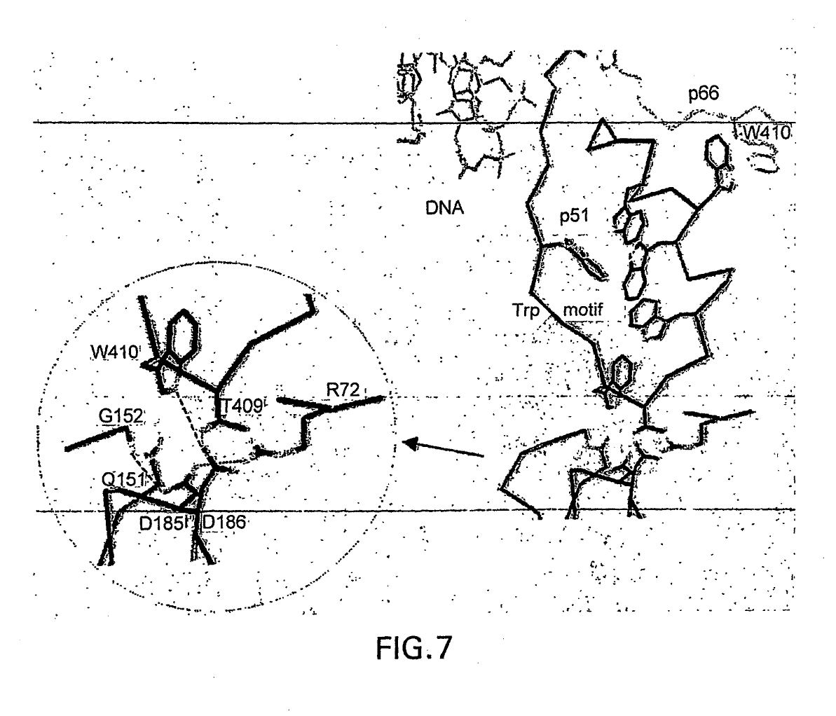

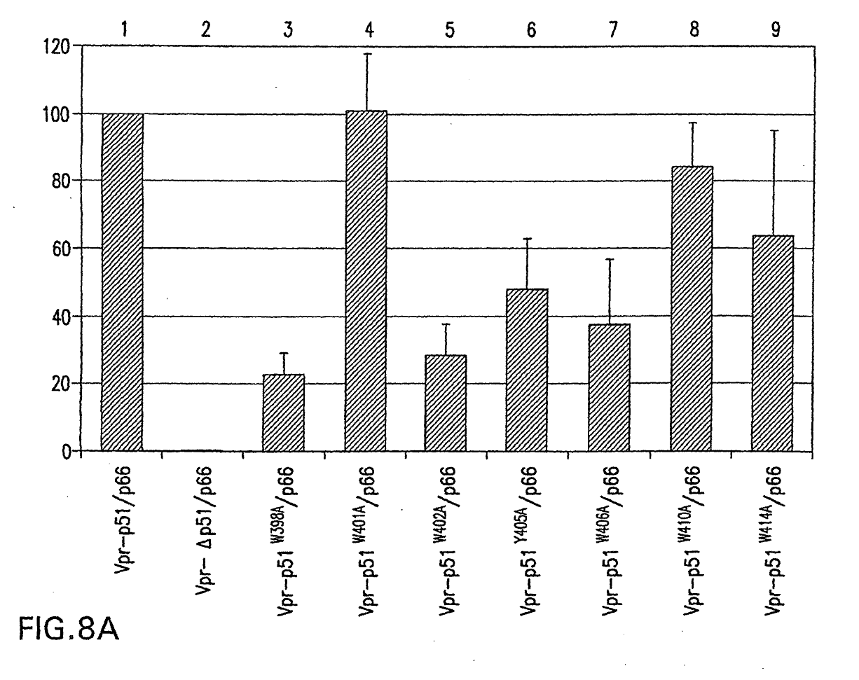

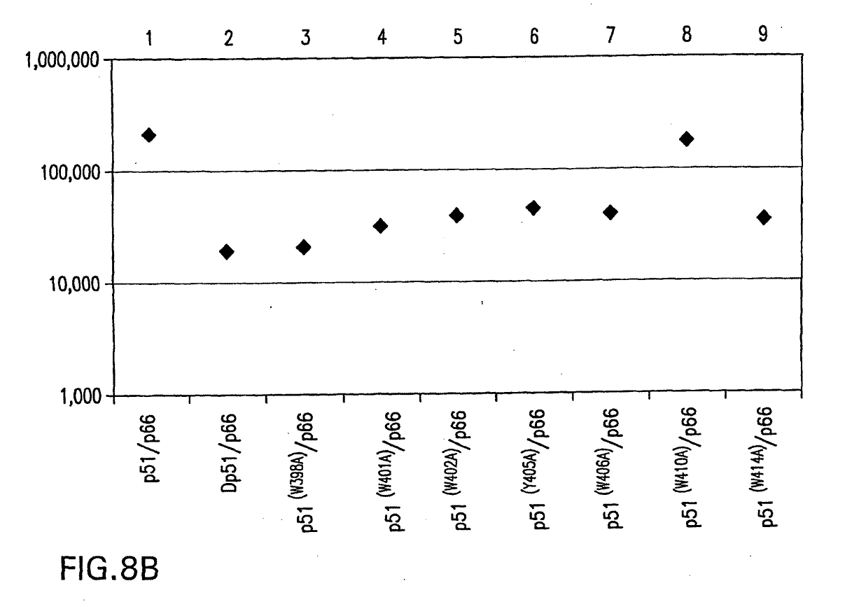

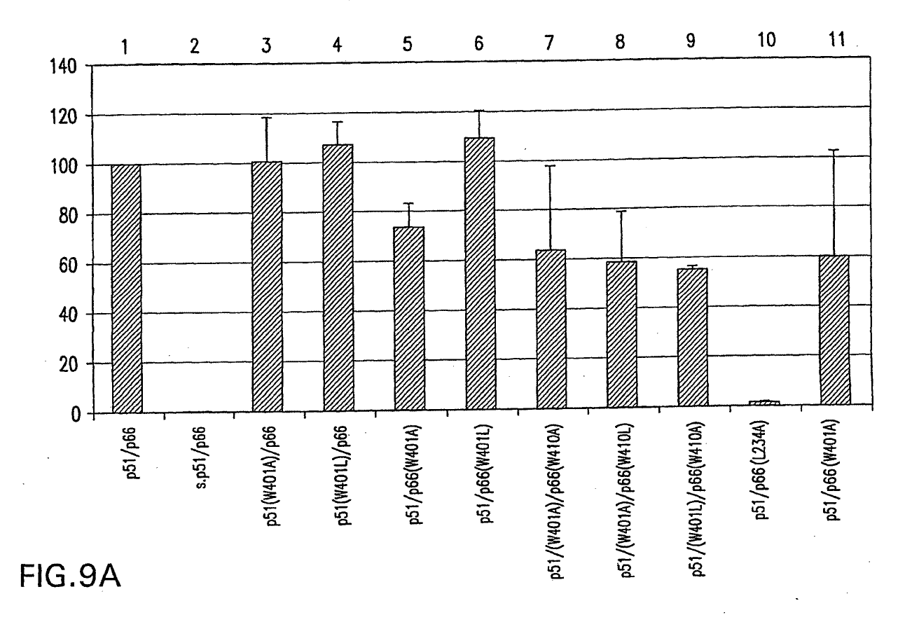

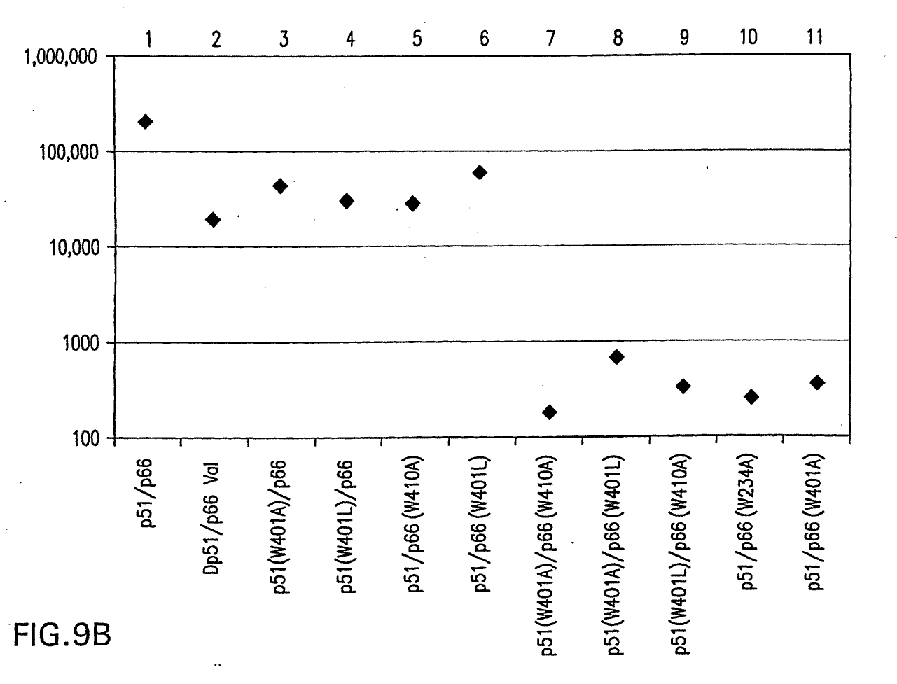

The D185 and D186 residues of p51 YMDD are within interacting distance (approximately 3 Å) of residues T409 and W410 of the p51 connection subdomain. The T409 and W410 residues lie in the loop between alpha helix L (αL) and beta sheet 20 (β20). This loop is a part of the putative “tryptophan motif” (Trp-motif) of the p51 connection subdomain, which is believed to be critical for p51-p66 dimerization (Baillon et al., 1991; Divita et al., 1994; Tachedjian et al., 2003). It is plausible that mutation at the p51 YMDD aspartates cause repositioning of the αL-β20 loop, which in turn could affect multiple interactions involving the Trp-motif and the heterodimer interface. The orientation of the αL-β20 loop could also influence template binding as these residues are in the proximity of the floor of the DNA binding cleft/RNase H primer grip, which includes K390, K395 and E396 of p51 that interact directly with the template-primer (Huang et al., 1998; Sarafianos et al., 2001). Mutation of the p51 YMDD aspartates may also affect intermolecular interactions that maintain its structure in the RT heterodimer. In the p51 subunit, the connection subdomain folds into the expanded cleft between its fingers and thumb subdomains, which gives it a “closed” conformation. Since the connection subdomain of p51 makes multiple contacts with the three other subdomains of p51 (fingers, palm and thumb), destabilization of the interaction between the αL-β20 loop and YMDD can have global effects on RT folding. In addition to the interactions with T409 and W410, the YMDD motif is buried within the core of p51, and thus, the aspartates could interact with other neighboring residues. These include interdomain interactions between D185/186 and R72 of the p51 fingers subdomain; D185 and Q151-G152 at the tip of α helix E in the palm subdomain and D185/D186 with the tryptophan-rich region of p51 (FIG. 7).

The finding that subunit-specific analysis of RT function can be studied using infectious virus has broad implications. While the p51 subunit was believed to function primarily as a scaffold to maintain the active structure of p66 (Hughes, 2001; Telesnitsky and Goff, 1997), other functions have been suggested, including involvement in tRNA primer-binding (Arts et al., 1994; Jacques et al., 1994), loading of p66 onto the template-primer (Harris et al., 1998) and enhancement of strand displacement (Amacker et al., 1995; Hottiger et al., 1994). The dimer interface between p51 and p66 is critical for reverse transcription and it has been proposed as an ideal target for therapeutic intervention (Divita et al., 1994; Morris et al., 1999; Restle et al., 1990). This notion was supported by several studies demonstrating that mutation of amino acid residues involved in subunit interactions alter the arrangement of the RT subdomains and disrupt RT function (Ghosh et al., 1996; Menendez-Arias et al., 2001; Tachedjian et al., 2003). Mutations in p51 have been also implicated in resistance to non-nucleoside reverse transcriptase inhibitors (NNRTIs) and inhibitors of RNase H activity. The E138K mutation, which confers resistance to TSAO {2′,5′-Bis-O-(tert-butyldimethylsilyl)-3′spiro-5″-(4″-amino-1″,2″-oxathiole-2″,2″-dioxide)} has been mapped to the p51 subunit (Jonckheere et al., 1994; Sluis-Cremer et al., 2000). The C280S mutation in RT causes resistance to the RNase H inhibitor N-ethylmaleimide (NEM) (Loya et al., 1997). Both the p51 and p66 subunits were found to contribute to the resistance of the enzyme to NEM in vitro.

Materials and Methods

Cells and Antibodies

The 293T, JC53 (Platt et al., 1998), and TZM-b1 cell lines (Wei et al., 2002) were maintained in Dulbecco's modified Eagle's medium (DMEM) supplement with 10% fetal bovine serum (FBS), penicillin (100 units/ml) and streptomycin (0.1 mg/ml). The anti-RT antiserum (R1465) was generated against HIV-1 RT expressed in E. coli. Briefly, the entire RT coding region of HIV-1/pSG3 was ligated into the prokaryotic pGEX expression vector (pGEX-RT). E. coli (DH5α) were transformed with pGEX-RT and protein expression was induced with isopropyl β-D-thiogalactopyranoside (IPTG). Expression of the glutathione S-transferase-(gst) RT fusion protein was confirmed by SDS-PAGE. Soluble gst-RT protein was purified and RT was released by thrombin cleavage using previously described procedures (Smith and Johnson, 1988). New Zealand white female rabbits were immunized subcutaneously with 200 μg of purified RT protein emulsified in an equal volume of Freund's complete adjuvant. Rabbits were boosted at two week intervals with 200 μg of RT mixed with an equal volume of Freund's incomplete adjuvant. Sera were tittered and analyzed for specificity by immunoblotting against purified preparations of both the immunizing protein and concentrated HIV-1 virions. Additional antibodies used in these studies included monoclonal antibodies to HIV-1 capsid (183-H12-5C) and HIV-1 RT (8C4 and 7E5.

HIV-1 Proviral Clones

The HIV-1 pSG3 proviral clone (SG3) (Ghosh et al., 1993) (Genbank Accession #L02317) was used to produce wild-type virus, and to construct RT deficient proviral clones and all recombinant RT and IN expression plasmids. The pSG3^{FN} (FN) clone was constructed using the strategy described by Dubay et. al. (Dubay et al., 1992) for the HXB2 pFN clone (FIG. 1B). Briefly, the FN clone contains an in-frame 110 amino acids deletion and was created by Acc65I digestion to remove a 330-nucleotide fragment of the pol gene. The 5′ overhang was filled using dGTP and the Klenow fragment of DNA polymerase. The remaining single-stranded regions were removed with S1 nuclease and the plasmid was religated. The deleted DNA segment encoded a large part of RNase H and 13 amino acids of the carboxyl-end of the polymerase domain of RT. This clone encodes a truncated form of RT while maintaining the IN coding region in-frame.

The pSG3^{M7} (M7) proviral construct was created from pSG3^{S-RT} (S-RT) (Wu et al., 1997). In addition to stop codons in the RT and IN coding regions of pSG3^{S-RT}, M7 contains three additional stop codons at amino acid positions 441 (TAA), 444 (TGA) and 447 (TAG) and a D443N RNase H catalytic mutation in the RNase H reading frame. The primers (sense [5′-AAGCCCGGGATGGATGGCCCAAAAGT-3′], SEQ ID NO: 10 and antisense [5′-TCCTAAACGCGTCTCCCTCTAAGCTGCTCAATTTACTTAGAAAGT-3′], SEQ ID NO: 11) containing XmaI and MluI sites, respectively, and the primers (sense [5′-ACTTTCTAAGTAAATTGAGCAGCTTAGAGGGAGACGCGTTTAGGA-3′] (SEQ ID NO: 12) and antisense [5′-TATGTCGACACCCAATTATGAAAAG-3′] (SEQ ID NO: 13)) containing MluI and SalI sites, respectively, were used to amplify two DNA fragments from the S-RT constructs (nucleotides 2132-3455 and 3410-5340). The XmaI-MluI and MluI-SalI PCR products were digested with corresponding restriction endonucleases, purified and ligated together into an XmaI-SalI cut pSG3^{S-RT} plasmid.

Construction of Heterodimeric RT Expression Plasmid

To express the RT subunits in trans with RT-minus proviral DNA, the pLR2P-vpr-p51-IRES-p66 (vpr-p51/p66) expression plasmid was constructed. Briefly, the sense [5′-TAGATCAGATCTGTTGACTCAGATTGGTTGCA-3′] (SEQ ID NO: 14) and antisense [5′-ATCTACACGCGTTTAGAAGGTTTCTGCGCCTT-3′] (SEQ ID NO: 15) primers containing the BglII and MluI restriction sites (underlined), respectively, were used to PCR amplify a p51-containing DNA fragment from pSG3. The internal ribosome entry site (RES) was PCR amplified from the encephalomyocarditis virus (EMCV) (Duke et al., 1992) (Genbank Accession #NC_{—}001479) using the sense ([5′-TTATTAACGCGTCCGCCCCTCTCCCTCCCCCC-3′] (SEQ ID NO: 16) and antisense [5′-CCATCCCGGGCTTTAATTTTACTGGTACAGTTTCAATAGGAC TAATGGGTCCCATGGTATTATCGTCTT-3′] (SEQ ID NO: 17) primers containing MluI and XmaI sites (underlined), respectively. The PCR-derived p51 fragment was digested with BglII and MluI, while the IRES fragment was digested with MluI and XmaI. These two fragments were ligated simultaneously into the BglII-XmaI-cut pLR2P-vprRT (Wu et al., 1997), generating pLR2P-vpr-p51-IRES-p66. This construction strategy (FIG. 1A) placed vpr and RT in-frame, while preserving the N-terminal protease cleavage (PC) site of RT by including 33 bps of PR sequence 5′ of RT. The antisense primer introduced a translational stop codon (TAA) to terminate RT expression at amino acids 440, which is the full-length p51 subunit. The vpr-p51 reading frame was followed by the IRES and then p66. To enable efficient expression of p66, an artificial Kozak sequence was included at the 5′ of the p66 coding sequence (Kozak, 1987). This modification added a Met-Gly onto the p66 N-terminus. The pLR2P-vpr-Δ51-IRES-p66 (vpr-Δp51/p66) control plasmid was constructed to contain a translational stop codon at the first amino acid position of p51 by amplification of a BglII-MluI DNA fragment from the S-RT clone. Other derivatives of vpr-p51/p66 were constructed using PCR based site-directed mutagenesis, restriction digestion with the appropriate enzyme, and cloning into the BglII-MluI or XmaI-XhoI sites for p51 or p66 mutant clones, respectively. All clones were confirmed by sequencing. The pLR2P-vprIN (vpr-IN) expression vector has been described previously (Wu et al., 1997).

Transfections and Analysis of Virus Infectivity

DNA transfections were performed on monolayer cultures of 293T cells grown in E-well plates using the calcium phosphate DNA precipitation method. Unless otherwise noted, each cell monolayer (well) was transfected with 6 μg of proviral DNA, 3 μg of the vpr-p51/p66 constructs and 1 μg of the vpr-IN constructs. Culture supernatants from the 293T cells were collected 60 h post-transfection, clarified by low-speed centrifugation (1000×g, 10 min), and filtered through 0.45 μm pore-size sterile filters. The clarified supernatants were analyzed for HIV-1 p24 antigen concentration by ELISA (Beckman-Coulter Inc.).

Virus infectivity was assessed using the TZM-b1 reporter cell line as described earlier (Wei et al., 2002). Briefly, virus containing supernatants were normalized for p24 antigen concentration, serially diluted (five-fold dilutions) and used to infect monolayer cultures of TZM-b1 cells. At 48 hrs post-infection, the cells were fixed and stained with 5-bromo-4-chloro-3-indolyl-β-D-galactopyranoside (X-gal) reagent as described earlier (Kimpton and Emerman, 1992). The blue-stained cells were counted using a light microscope. Wells containing between 30 and 300 blue cells were used to calculate the infectious units of virus per ng of p24 antigen (IU/p24-ng).

Semiquantitative Detection of Viral DNA

The PCR method used to analyze the synthesis of nascent viral DNA in infected cells was similar to those described earlier (Fassati and Goff, 2001; Zack et al., 1990). Briefly, 500-ng equivalents (p24 antigen) of transfection-derived virions were incubated with DNase I (4 μg/ml; Worthington Inc.) at 37° C. for 1 hr to minimize plasmid DNA carryover. The treated virus was then used to infect one million JC53 cells for 4 hrs in DMEM (1% FBS, 10 μg/ml DEAE-dextran). The cells were washed twice with DMEM, and the medium was replaced with complete DMEM (10% FBS). At 18 hrs post-infection, the cells were lysed and total DNA was extracted by organic methods, resuspended in 200 μl of distilled water and treated with the DpnI restriction endonuclease to digest bacterially derived plasmid DNA. Each PCR subjected 250 pgs of DNA extract to 30 rounds of amplification with primers designed to detect early (R-U5 [sense nucleotides 79-99 AGCTTGCCTTGAGTGCTTCAA (SEQ ID NO: 18) and antisense nucleotides 182-157 CTGCTAGAGATTTTCCACACTGACTA] SEQ ID NO: 19) and late (R-gag [sense nucleotides 43-63 GGCTAGCTAGGGAACCCACTG (SEQ ID NO: 20) and antisense nucleotides 355-334 ATACTGACGCTCTCGCACCCAT] (SEQ ID NO: 21)) viral DNA. The PCR products were separated on a 1.0% agarose gel and visualized by ethidium bromide staining. The relative amount of amplified DNA was determined by comparison to known standards (serial dilutions of pSG3 DNA).

Western Blot (Immunoblot) Analysis

Transfection-derived virions were concentrated by ultracentrifugation through 20% sucrose cushion (125,000×g, 2 hr, 4° C.) using a SW41 rotor (Beckman Inc.). Pellets were solubilized in loading buffer (62.5 mM Tris-HCl [pH 6.8], 0.2% SDS, 5% 2-mercaptoethanol, 10% glycerol), boiled, and proteins were separated on 12.0% polyacrylamide gels containing SDS. Following electrophoresis, proteins were transferred to nitrocellulose (0.2-μm pore size) by, electroblotting and incubated for 1 hr at room temperature in blocking buffer (5% nonfat dry milk in phosphate-buffered saline [PBS]). The blocked blot was exposed to the appropriate primary antibody for 1 hr in blocking buffer with constant mixing. After extensive washing, bound antibodies were detected by chemiluminescence using horseradish peroxidase-conjugated species-specific secondary antibodies (Southern Biotechnology Associates, Inc.) as described by the manufacturer (Amersham Biosciences).

### Example 2

### Exogenous Reverse Transcriptase Assay

Disclosed are biochemical assays for determining reverse transcriptase activity. One example of such an assay is the Chemiluminescent Reverse Transcriptase Assay by Roche (Cat. No. 1 828 657, Instruction Manual Version 3, February 2004, herein incorporated by reference in its entirety for its teaching regarding reverse transcriptase assays). The protocol is a non-radioactive enzyme immunoassay useful for highly sensitive, quantitative determination of reverse transcriptase activity by chemiluminescence detection. This assay is designed for highly-sensitive and quantitative determination of RT activity, e.g. in cell cultures and other life science research applications. The assay has been shown to be useful for the determination of RT activity derived from a variety of retroviruses, including HIV-1, HIV-2, SIV-1 and CAEV. The assay can be used to determine the propagation of retroviruses in retrovirus-infected mammalian cell cultures. The assay can also be used for in vitro screening for RT inhibitors.

### Example 3

### p51-IRES-p66 Rescues the Infectivity of RT-IN-Minus Virus (M7)

Regarding Table 4: the table shows that Vpr-p51-ires-p66 rescues the infectivity of RT-IN-minus virus (M7), and viruses derived from proviral DNA containing mutations in the YMDD motif of RT, including YMAA and YMND. Virus derived from proviral DNA and the control pLR2P-vpr is not infectious. Methods: 293T cells were transfected with the indicated plasmid (either viral DNA or trans-RT DNA) DNAs plus the pLR2P-vor-IN expression plasmid DNA. 48 hrs later the supernatant viruses were collected and analyzed for infectivity using the JC53-BL reporter assay.

TABLE 4

| | | Blue | Infectious | p24 | | |
|---|---|---|---|---|---|---|
| Construct | | Cells | virions/ml | ngs/ml | Virions/ng | % of |
| Name | | A | A/B × 1000 = C | D | C/D | SG3 |
| pLR2P-vpr | — | 93 | #DIV/0! | 714 | #DIV/0! | 100.00 |
| pLR2P-vpr | — | 0 | 0.00E+00 | 397 | 0.00E+00 | 0.00 |
| pLR2P-vpr | — | 0 | #DIV/0! | 688 | #DIV/0! | #DIV/0! |
| vpr-p51-IRES- | — | 66 | #DIV/0! | 563 | #DIV/0! | #DIV/0! |
| p66 | | | | | | |
| pLR2P-vpr | — | 0 | #DIV/0! | 382 | #DIV/0! | #DIV/0! |
| vpr-p51-IRES- | — | 21 | #DIV/0! | 1072 | #DIV/0! | #DIV/0! |
| p66 | | | | | | |

| | | Blue | | | p24 | | |
|---|---|---|---|---|---|---|---|
| Construct | | Cells | Dilution | Virions/ml | ngs/ml | Virions/ng | % of |
| Name | | A | B | A/B × 1000 = C | D | C/D | SG3 |
| pLR2P-vpr | — | 225 | 0.2 | 1.13E+06 | 714 | 1.58E+03 | 100.00 |
| pLR2P-vpr | — | 0 | 5 | 0.00E+00 | 397 | 0.00E+00 | 0.00 |
| pLR2P-vpr | — | 2 | 5 | 4.00E+02 | 688 | 5.81E−01 | 0.04 |
| vpr-p51-IRES- | — | 843 | 5 | 1.69E+05 | 563 | 2.99E+02 | 19.01 |
| p66 | | | | | | | |
| pLR2P-vpr | — | 0 | 5 | 0.00E+00 | 382 | 0.00E+00 | 0.00 |
| vpr-p51-IRES- | — | 66 | 5 | 1.32E+04 | 1072 | 1.23E+01 | 0.78 |
| p66 | | | | | | | |

### Example 4

### The Tryptophan Motif of HIV-1 Reverse Transcriptase Structural Analysis of the Putative RT Dimerization Domain (Tryptophan Motif)

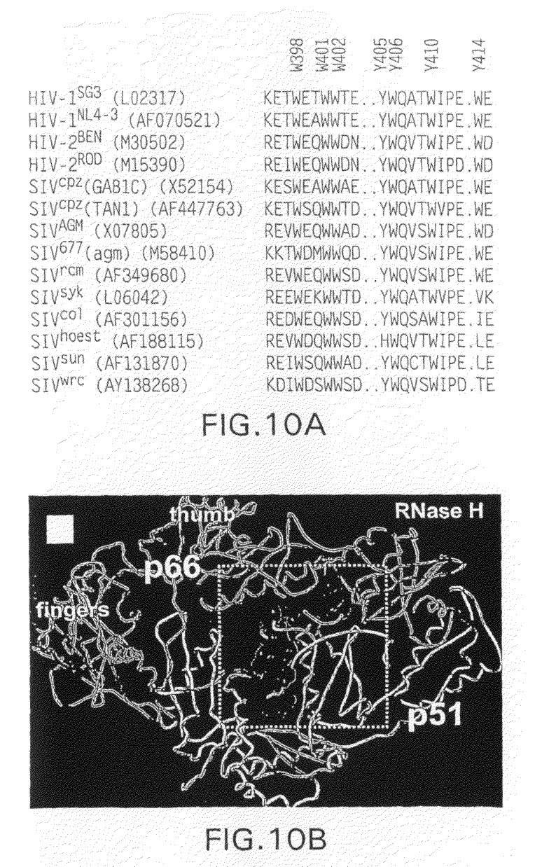

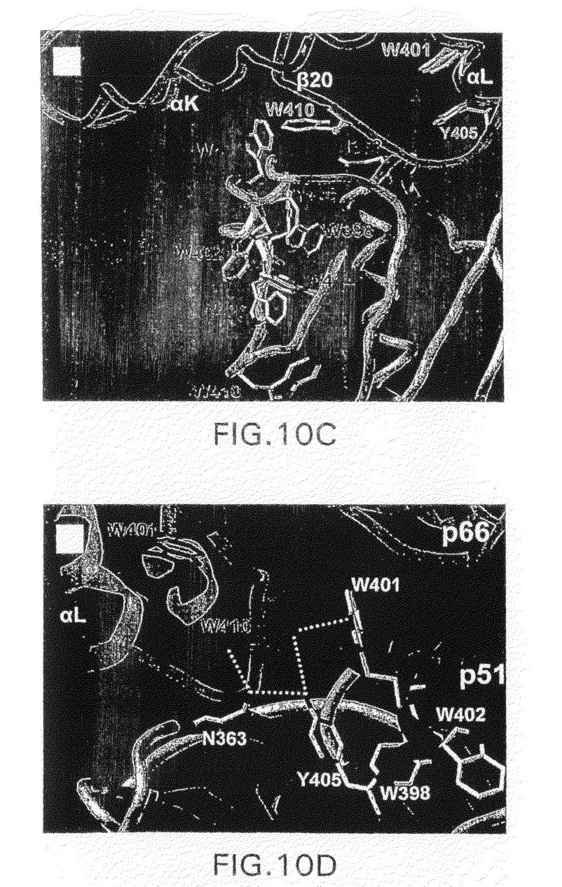

The Trp-motif of HIV-1 is comprised of aromatic amino acids in the connection subdomain (between amino acid positions 398 and 14). Alignment analysis shows that the six tryptophans (W398, W401, W402, W406, W410 and W414) and tyrosine (Y405) residues are conserved within the connection subdomain of most primate lentiviruses (FIG. 1A). The most conserved residue amongst all the lentiviruses is W398. To understand the interactions at the dimerization interface between the two connection subdomains of HIV-1 RT, several crystal structures of HIV-1 RT, including unliganded (1DLO), were compared in complex with substrates (pdb codes 1TO5, 1RTD, 1HYS, 1N6Q) or NNRTIs (1HNI, 1SV5, 1S6P, 1S9E, 1DTT, 1BMQ, 1FK9). The overall structure of the dimerization interface is conserved among the various RT complexes. Stabilization of the heterodimer involves direct, as well as indirect interactions between residues from the two subunits. Specifically, a key direct interaction appears to involve three p51 residues from the β18-αK (N363) loop, the αL helix (W401), and the αL-β20 loop (Y405), that are within interacting distance of residue W410 located in the αL-β20 loop of p66 (FIG. 10). In addition to these interactions, the W401 in p51 is also within interacting distance with p66 residue P412 at the base of the β20-sheet in p66. Indirect interactions can also play a role and involve residues that are proximal to the p66 or the p51 part of the interface. In the p51 subunit, a cluster of four Trp residues (W398, W402, W406 and W414) is proximal to the p51 interface residues (Y405, N363, and W401) (FIG. 10).

Expression and Virion Incorporation of Hetromeric RT Containing p51 Trp-Motif Mutations

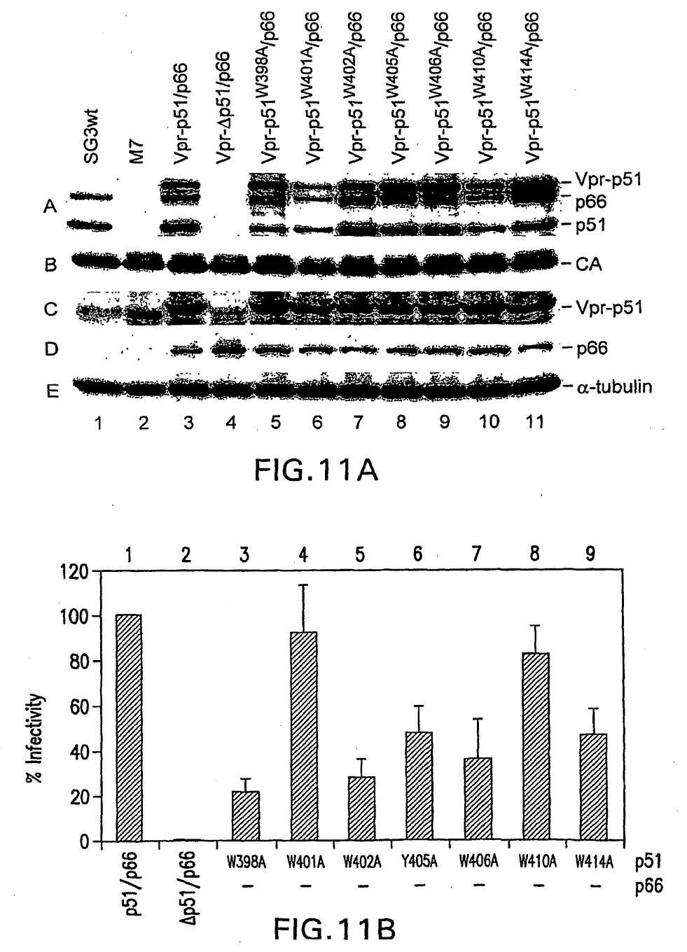

The p51 Trp-motif residues were independently mutated to alanines in the pLR2P-vpr-p51-IRES-p66 (vpr-p51/p66) expression plasmid. Wildtype and each of the mutant DNAs were cotransfected into 293T cells with the RT-IN defective M7 proviral DNA, and progeny virions were examined by immunoblot analysis. The control RT-IN-minus M7 particles (FIG. 2A, lane 2) did not contain RT. A similar level of both RT subunits (p51 and p66) was detected in particles derived by cotransfection of M7 and the wildtype vpr-p51/p66 expression plasmid (lane 3). The pLR2P-vpr-Δp51-IRES-p66 (vpr-Δp51/p66) control plasmid does not express p51, and the absence of detectable p66 (lane 4) confirmed that its incorporation was dependent on the expression of p51 (Vpr-p51). Analysis of p51^{W398A}/p66 (lane 5) showed p51 and p66 in the virion, however, an additional band was also detected migrating just below p66. This band was confirmed to be a product of the p66 subunit by probing with mAb (7E5) specific to the RNase H domain. To a lesser extent, a similar p66 product was also seen in some of the other p51 mutants. Notably, the aberrant p66 product seemed to associate with mutants of residues (W398A, W402A, W406A and W414A) that cluster together proximal to the heterodimer interface (FIG. 10C). Wildtype HIV-1 SG3 virions were analyzed as an additional control (lane 1). Immunoblot analysis using mAb to CA confirmed that approximately the same amount of each virus was analyzed (FIG. 11B). Examination of transfected cells by immunoblotting with polyclonal anti-Vpr serum (FIG. 11C) demonstrated that all of the mutants expressed Vpr-p51 (lanes 5-11) at levels similar to that of wildtype Vpr-p51 (lane 3). A replica blot probed with 7E5 mAb confirmed that p66 was expressed at a similar level among the transfected cells (FIG. 11D). The amount of cellular protein analyzed was similar, as demonstrated by probing for the α-tubulin protein (FIG. 11E).

To examine if the aberrant p66 was due to misprocessing by the viral protease (PR), 293T cells were cotransfected with a PR-defective proviral DNA (PR catalytic mutant: D25A) and vpr-p51^{W398A}/p66. Detection of the aberrant p66 product in these virions (data not shown), suggested that it was generated independently of the HIV-1 PR.

Functional Analysis of p51 Trp-Motif RT Mutants

The functionality of the p51 Trp-motif mutants was analyzed in a single-round infectivity assay, using the TZM-b1 reporter cell line (Wei et al. Antimicrob. Agents Chemother. 46:1895-905 (2002)). Virions were generated by cotransfecting 293T cells with M7, wildtype or mutant vpr-p51/p66 and vpr-IN. vpr-IN was included since M7 lacks IN, which is required for efficient initiation of reverse transcription and for integration of the nascent viral cDNA (Mulky et al. (2004); Wu et al. J. Virol. 73:2126-35 (1999)). The infectivity of virions containing the wildtype trans-RT (Vpr-p51/p66) was normalized to 100% (FIG. 11F, lane 1). The infectivity of M7 derived by cotransfection with vpr-Δp51/p66 was less than 0.2% compared to vpr-p51/p66 (lane 2). Mutations in the tryptophan cluster (W398A, W402A, W406A and W414A) decreased infectivity to less than 50% (lanes 3, 5, 7 and 9, respectively), with the W398A mutant being the most defective. The infectivity of the p51^{W401A}/p66 mutant (lane 4) was similar to that of wildtype, while the p51^{Y405A}/p66 mutant (lane 6) was reduced to about 50%. The p51^{W410A}/p66 mutant (lane 8) had little effect on infectivity.

Subunit-Specific Mutagenesis of Trp-Motif Residues at the Heterodimer Interface

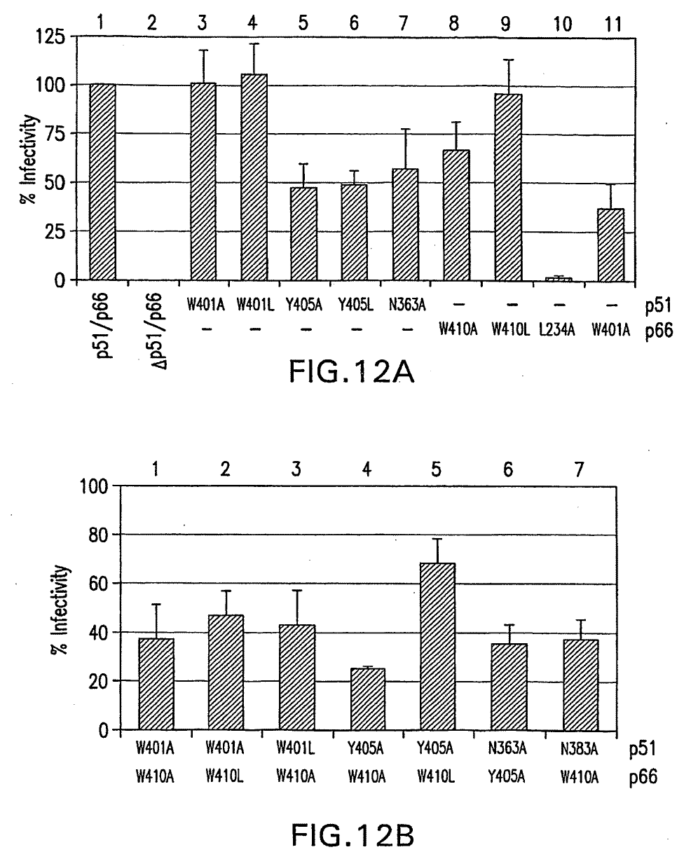

The analysis of inter-subunit interactions was focused initially on mutagenesis of individual residues to either alanine or leucine. The infectivity of the wildtype RT trans-heterodimer was normalized to 100% (FIG. 12A, lane 1). The vpr-Δp51/p66 was less than 0.2% infectious (lane 2). Replacement of p51^{W401} with either alanine or leucine did not affect viral infectivity (lanes 3 and 4). Both the p51^{Y405A}/p66 and p51^{Y405L}/p66 mutant RTs reduced infectivity to approximately half of that of the wildtype trans-heterodimer (lanes 5 and 6, respectively). Mutation of N363 in p51 to alanine also reduced infectivity, albeit to a lesser extent than the 405 mutations (lane 7). The replacement of p66^{W410} with alanine caused a slight reduction in infectivity (lane 8), while the leucine substitution had no effect (lane 9). The p51/p66^{L234A} and p51/p66^{W401A} mutants, reported previously as mutations that affect dimer formation, were included as controls in the experiments (Tachedjian et al. (2003), Ghosh et al. Biochemisty 35:8553-62 (1996)). The p51/p66^{L234A} mutant reduced infectivity to less than 5% (lane 10), while the p51/p66^{W401A} mutant was approximately 40% infectious (lane 11).

To further delineate these Trp-motif interactions, residues that lie within interacting distance of each other were mutated in pairs. Mutations were made in conjunction at residues W401 and W410 of p51 and p66, respectively. Infectivity analysis of p51^{W401A}/p66^{W410A}, p51^{W401A}/p66^{W410L} and p51^{W401L}/p66^{W410A} showing that mutagenesis of both residues together reduced viral infectivity (approximately 40%) to a significantly greater extent compared to that of the single mutations (FIG. 12B, lanes 1, 2 and 3). Analysis of RT containing simultaneous mutations at p51^{Y405} and p66^{W410} indicated that substitution of both residues with alanine decreased infectivity to about 25% of the wildtype heterodimer (lane 4). In contrast, the infectivity of the p51^{Y405A}/p66^{W410L} double mutant (lane 5) was comparable to that of the p51^{Y405A}/p66 single mutant, showing that mutagenesis of p66^{W410} to leucine does not affect its interaction with Y405 of p51. The model predicted that the residue N363 in p51 interacts with both p51^{Y405} and p66^{W410}. The p51^{N363A;Y405A}/p66 (lane 6) and p51^{N363A}/p66^{W410A} (lane 7) virions had similar infectivity, which was reduced to approximately 35% of wildtype and substantially lower than the respective single mutants. Immunoblot analysis detected only a slight reduction in Vpr-p51-mediated p66 incorporation in some of the double mutants.

Analysis of the Inter-Subunit Interface in Provirus

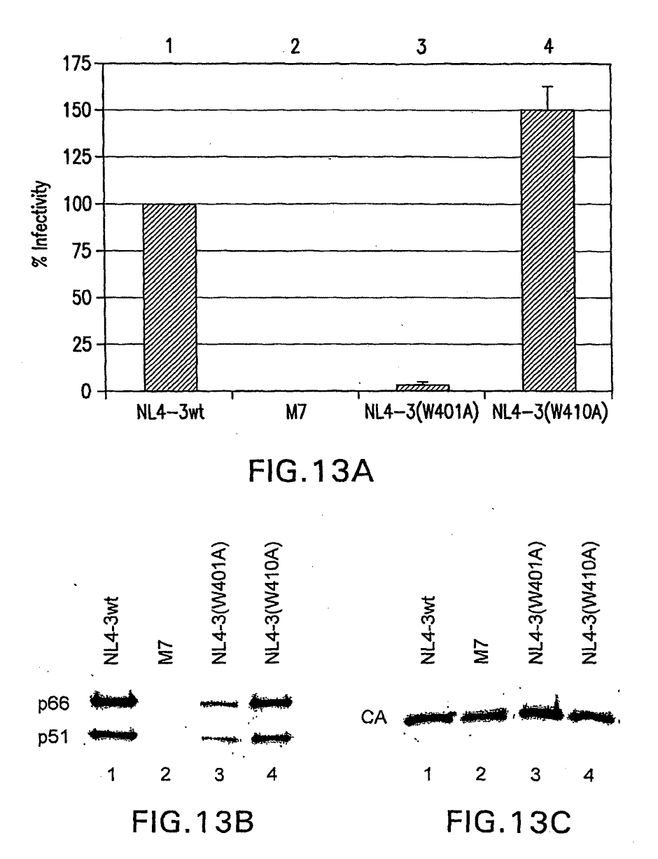

The results indicated an interaction at the dimer interface between residues p51^{W401} and p66^{W410} that is important for subunit interaction. Additional analysis of the W401A and W410A mutations was conducted in the context of the complete HIV-1 NL4-3 proviral clone. The wildtype or mutant proviral DNAs were transfected into 293T ceils and progeny virions were analyzed for infectivity. The infectivity of virus containing the W401A mutation was less than 5% of that of wildtype (FIG. 4A, lanes 1 and 3). In contrast, W410A caused an increase in virus infectivity (lane 4). The non-infectious RT-minus M7 clone was included as negative control (lane 2). Notably, immunoblot analysis showed a significantly reduced amount of the W401 mutant RT in virions, compared to either the wildtype or W410A mutant (FIG. 13B). Probing a replica blot with mAb to CA confirmed that approximately the same amount of each virus was analyzed (FIG. 13C).

Analysis of W401A Mutation in the RT Trans-Heterodimer

To determine the effect of the W401A proviral DNA mutation on RT, the mutation was analyzed by subunit-specific trans-complementation, wherein the mutation was present p51, p66 or both p51 and p66. The infectivity of virions complemented with the wildtype trans-heterodimeric RT was normalized to 100% (FIG. 5A, lane 1). Subunit-specific mutagenesis of p51 (p51^{W401A}/p66) did not significantly affect viral infectivity, as described above (lane 3), while mutagenesis of the p66 subunit (p51/p66^{W401A}) reduced infectivity to about 40% (lane 4). The effect of this mutation in both subunits (p51^{W401A}/p66^{W401A}) was quite dramatic (lane 5), reducing infectivity to levels similar to that observed for the W401A mutant provirus.

Analysis of virions produced by coexpressing vpr-p51^{W401A}/p66 demonstrated, as expected, wildtype levels of both subunits (FIG. 5B, lanes 1 and 3). However, when the W401A mutation was present in the p66 subunit (vpr-p51/p66^{W401A}), the incorporation of p66 into M7 virions was reduced (lane 4). Interestingly, the presence of W401A in both p51 and p66 further reduced the amount of p66 detected in virions (lane 5). In all cases, reduced virion p66 was also seen using a polyclonal RT antiserum and the amount of p66 expressed in the cells was normal (data not shown). The decrease in virion p66 observed with the p51/p66^{W401A} and p51^{W401A}/p66^{W401A} mutants was identical to that observed when virions were produced using a PR-defective proviral DNA (PR catalytic mutant: D25A) in place of M7. This result confirmed that less p66 was detected in the M7 virions because the W401A mutation(s) impaired p66 virion incorporation. Importantly, this result ruled out the possibility that less p66 was detected due to overprocessing and conversion of p66 to p51 by the viral protease subsequent to virion assembly.

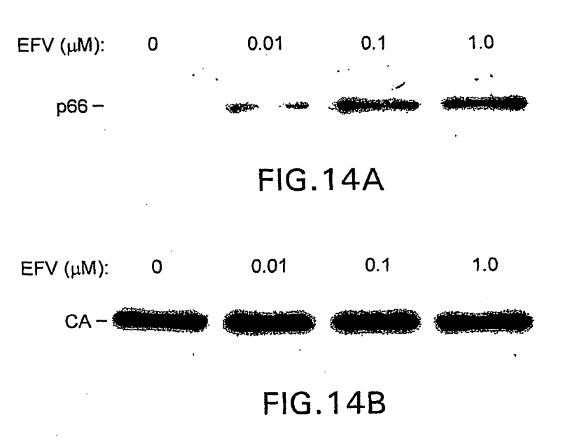

To analyze the infectivity of trans-RT complemented virions in a target cells that is more physiologically relevant, the JLTRG-R5 reporter cell line was used. These cells are derived from JLTRG cells and are of Jurkat T cell lineage. (Ochsenbauer-Jambor et al. submitted (2004); Kutsch et al. Antimicrob. Agents Chemother. 48:1652-63 (2004)). The JLTRG-R5, cells have stably integrated EGFP reporter under control of the HIV-1 LTR, and thus EGFP expression is induced by virus infection. Infectivity for the wildtype trans-RT heterodimer was normalized to 100% (FIG. 14C, lane 1). The Δp51/p66 exhibited infectivity below 5% (lane 2). The W401A mutation in p51 did not affect viral infectivity (lane 3), while W401A in p66 reduced infectivity to about 50% of that of the wildtype trans-RT (lane 4). The presence of W401A simultaneously in p51 and p66 significantly decreased infectivity (lane 5). These results were consistent with those generated using the TZM-b1 assay, which was used for analysis in a parallel experiment. The ability to analyze virions containing trans-heteromeric RT using a T cell line emphasizes the biological relevance of our approach. The results indicate that the trans-RT heterodimer complemented virions can be analyzed in multiple human-derived reporter cell lines including more physiologically relevant T cell lines.

Efavirenz Enhances Subunit Interaction in the Trans-RT W401A Double Mutant

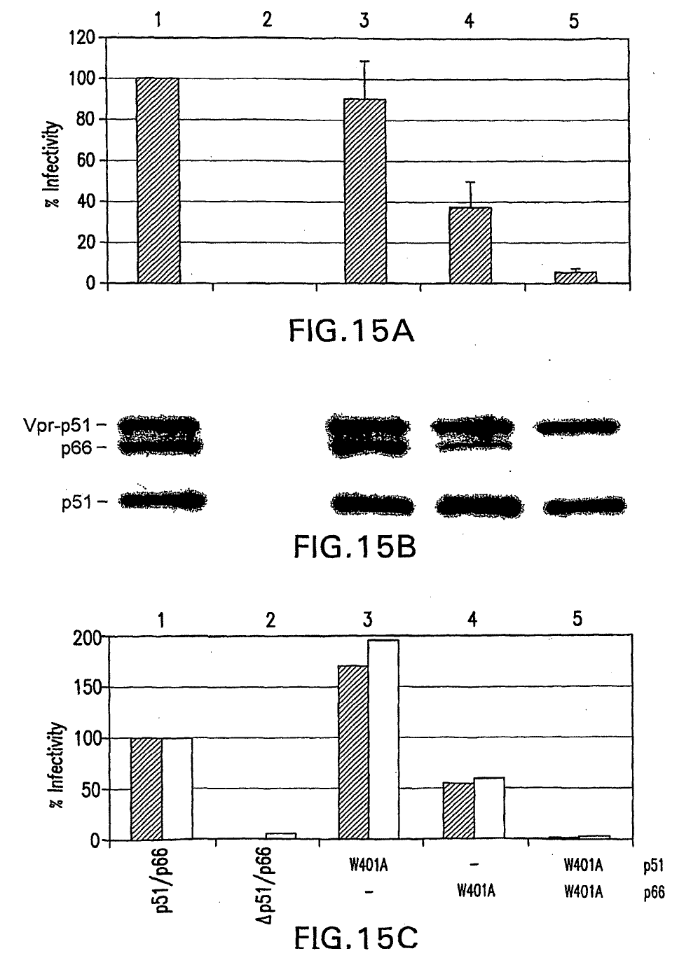

To examine the effect of dimerization enhancing drugs on the dimerization-defective W401A mutant trans-heterodimeric RT, EFV was added to the producer cells (transfected 293T cells) at concentrations ranging from 0.01-1.0 μM. Examination of virion-associated p66^{W401A} incorporation, which is dependent upon interaction with p51^{W401A} (Vpr-p51^{W401A}), showed that EFV rescued subunit dimerization in a dose-dependent manner (FIG. 15A). Equal virion protein loading was confirmed by probing a replica blot with anti-CA mAb (FIG. 15B). The amount of virion-associated p51 and Vpr-p51 was equal in both the absence and presence of drug. Similar results were also observed for other second generation NNRTIs.

Discussion

Two significant problems have heretofore hindered structure/function studies of RT using infectious virus. First, RT is encoded and assembled into virions as part of the Prl60^{Gag-Pol} polyprotein, and consequently, mutations in RT/Prl60^{Gag-Pol} can be pleiotropic, affecting multiple steps of the viral life cycle such as assembly, maturation, etc. (Yu et al. Virology 219:29-36 (1996), Quillent et al. Virology 219:29-36 (1996), Olivares et al. (2004), Tomonaga et al. Arch. Virol. 143, 1-14 (1998)). Analogous with the results for the W401A proviral mutant, Yu et al. have reported that mutations in the polymerase primer grip decrease virion-associated RT due in part to premature Gag-Pol processing (Yu et al. Virology 219:29-36 (1996)). The second problem was due to the heterodimeric nature of the RT. The asymmetry of the p51 and p66 subdomains entails that a mutation in one subunit is structurally and functionally non-equivalent to the same mutation in the other subunit. Thus a novel trans-complementation approach for analyzing the RT heterodimer in precise was developed molecular detail in the context of infectious virions. By exploiting this approach, several relevant questions concerning HIV-1 RT biology have been answered that were previously experimentally inaccessible. Primarily, these include (i) the role of hydrophobic, amino acid residues comprising the Trp-motif for subunit interaction and RT function, (ii) the contribution of amino acids at the p51/p66 connection subdomain interface to RT dimerization and virus infection, and (iii) the availability of a biologic approach capable of assessing the effects of both dimerization enhancing and disrupting drugs.

Structural analysis of interactions in the Trp-motif with residues at the subunit interface in several complexes of RT with substrate or inhibitors shows that the side-chain of W410 in the p66 αL-β20 loop, consistently within interacting distance of p51 residues W401, Y405, and N363 (FIG. 1D). trans-complementation analysis of these putative inter-subunit interactions showed that mutation of individual residues at this interface caused a measurable decrease in virus infectivity. Simultaneous mutagenesis of two inter-subunit residues within interacting distance of each other further impaired viral infectivity, showing that this effect was due to effects on subunit interactions. The data from immunoblot indicate similar to wildtype (Vpr-p51/p66) levels and processing of the mutant trans-RT of the two subunits.

The most severe effect on heterodimerization was observed for the p51^{W401A}/p66^{W401A} mutant. The presence of W401A in both subunits markedly impaired p51-p66 interaction, directly evidenced by a significant decrease in Vpr-p51^{W401A} mediated p66^{W401A} packaging. Based on this data, the structural analysis of several RT crystal structures and previous reports on the Trp-motif, repositioning the αL-β20 loop by mutating p66^{W401} and disruption of interactions involving p51^{W401,} p51^{W405}, p51^{N363} and p66^{W401} account for the findings. Subunit-specific mutational analysis of the W401 RT mutants demonstrates that W401 of the p66 and p51 subunits has distinct structural roles in the stabilization the RT heterodimer. In p51 the W401A mutation appears to affect the interactions at the interface, through disruption of the π-π interactions with p66^{W410}. However, the p66-W401A mutation affects the folding of the p66 subunit because it is at the interface of two helices (αL and αK) (FIG. 1C). Hence, when both subunits are mutated the different effects appear additive.

Structural analysis of the dimer interface in several RT crystal structures highlighted the potential importance of a cluster of four tryptophans in p51 (W398, W402, W406 and W414) proximal to the dimer interface. While these four p51 tryptophans do not directly interact with p66 residues, they are clustered together through hydrophobic interactions and seem poised to indirectly affect the dimer interface by their proximity to residues Y405, W401, and N363 of p51 that are at the p51-p66 interface (FIGS. 10B and 10C). Subunit-specific mutagenesis of these residues suggests that the Trp cluster affects the interaction between p51 and p66 (FIG. 11). Alanine substitution resulted in a misprocessed form of p66 that was detected in virions, trans-complementation analysis using PR defective virus indicates that a cellular protease is responsible for the aberrant processing of p66. The p51 Trp mutants can interact with and incorporate into virions a smaller processed form of p66 generated in the cell. Failure to detect the aberrant p66 in cell lysates, suggests it is present at a significantly lower level than wildtype p66. Alternatively, these p51 Trp mutants might form unstable heterodimers in which p66 is misfolded and thus, susceptible to proteolytic processing by a cellular protease. If this were true, it is interesting to note that dimer instability causes p66 misprocessing instead of normal processing to generate p51, which can associate with disassociated p66 to give functional RT heterodimer. The defect in infectivity seen with the mutants containing misprocessed p66 further supports this interpretation. Although p66 misprocessing seems to occur as a consequence of the atypical manner in which RT is expressed via the trans-complementation approach, it appears that residues W398, W402, W406 and W414 are important for proper RT subunit interactions.

### Example 5

### Materials and Methods

Cells, Antibodies and Antiviral Drugs

The 293T, JC53 (Platt et al. J. Virol. 72:2855-64 (1998), and TZM-b1 cell lines (Wei et al. Antimicrob. Agents Chemother. 46:1895-905 (2002) were maintained in Dulbecco's modified Eagle's medium (DMEM) containing 10% fetal bovine serum (FBS), penicillin (100 units/ml) and streptomycin (0.1 mg/ml). The JLTRG-R5 cell line 34 was maintained in Roswell Park Memorial Institute (RPMI) 1640 medium containing 15% FBS and gentamycin (0.1 mg/ml). Antibodies used included polyclonal anti-RT and anti-Vpr sera (Wu et al. J. Viroi. 69:389-98 (1995) and mAbs to human α-tubulin (Sigma), HIV-1 CA (183-H12-5C) and HIV-1 RT and RNase H (8C4 and 7E5), respectively, obtained through the NIH AIDS Research and Reference Reagent Program, Division of AIDS, NIAID, NIH.

Plasmid Constructs

The HIV-1 pSG3 (SG3) proviral clone (Genbank Accession #L02317) (Ghosh et al. Virology 194:858-64 (1993)) was used to produce wildtype virus, and to construct the RT-deficient proviral clone and all recombinant RT and IN expression plasmids (for abbreviations of plasmids see Table 3). The pSG3 (FN) clone was constructed using the strategy described by Dubay et al. (J. Virol. 66:6616-25, 1992) for the HXB2 pFN clone (FIG. 1b). Briefly, the FN clone contains an in-frame 110 amino acids deletion and was created by Acc65I digestion to remove a 330-nucleotide fragment of the pol gene. The 5′ overhang was filled using dGTP and the Klenow fragment of DNA polymerase. The remaining single-stranded regions were removed with S1 nuclease and the plasmid was ligated. The deleted DNA segment encoded a large part of RNase H and 13 amino acids of the carboxyl-end of the polymerase domain of RT. This clone encodes a truncated form of RT while maintaining the IN coding region in-frame. The RT-IN-minus pSG3^{M7} (M7) proviral construct was used for trans-complementation analysis with all the pLR2P-based RT and IN expression plasmids. For expressing the RT subunits in trans, the pLR2P-vpr-p51-IRES-p66 (vpr-p51/p66) plasmid 31 was modified by including 135 by of PR sequence 5′ of RT. This increased the molecular weight of the Vpr-RT fusion protein and enabled visual separation from p66 by immunoblot analysis. Briefly, p51-encoding sequence was PCR amplified from SG3 using primers containing BglII and MluI restriction sites, respectively. The internal ribosome entry site (IRES) was PCR amplified from the encephalomyocarditis virus (EMCV) (Genbank Accession #NC_{—}001479) with primers containing MluI and XmaI sites, respectively. The p51 and IRES DNA fragments were digested with corresponding endonucleases and ligated simultaneously into the BglII-XmaI cut pLR2P-vprRT 37. The vpr-p51/p66 was modified in that the N-terminal protease cleavage (PC) site of RT was maintained by including 135 bps of PR-encoding sequence 5′ ot-RT compared to 33 bps of PR sequence in the original construct. The vpr and p51 coding sequences were placed in-frame, with a translational stop codon (TAA) to terminate RT expression at amino acids 440, which is the full-length p51 subunit. The vpr-p51 reading frame was followed by the IRES and then the p66-encoding DNA sequence. Mutant derivatives of vpr-p51/p66 (Table III) were constructed using PCR-based site-directed mutagenesis and cloning into the Bg1II-M1uI or XmaI-XhoI sites for p51- and p66-containing DNA fragments, respectively. The pLR2P-vpr-Δp51-IRES-p66 (vpr-Ap51/p66) control expression plasmid was constructed to contain a translational stop codon at the first amino acid position of p51 (Mulky et al. (2004)). This plasmid controls for non-specific incorporation of p66 into viral particles. All mutant clones were confirmed by nucleotide sequence analysis. The pLR2P-vprIN(vpr-IN) expression vector has been described previously (Wu et al. EMBO J. 16:5113-22 (1997)).

Construction of Heterodimeric RT Expression Plasmids.

The pLR2P-vpr-p51-IRES-p66 (vpr-p51/p66) plasmid was constructed for independent expression of the RT subunits in trans. Since the molecular mass of the unprocessed Vpr-p51 fusion protein is very similar to that of p66, these two proteins are not distinguishable using antibody directed to the polymerase domain of RT. To allow differentiation between Vpr-p51 and p66 by molecular mass, three derivatives of the original vpr-p51/p66 construct were made. These constructs were generated by including additional PR sequence 5′ of the p51-coding region in vpr-p51. Either 90, 120 or 150 bps of PR sequence (encoding 30, 45 and 60 amino acids, respectively) were introduced at this position, generating vpr-^{30Pro}p51/p66, vpr-^{45Pro}p51/p66 and vpr-^{60Pro}p51/p66, respectively. The vpr-p51/p66-IN was constructed by cutting the pLR2P-vpr-RT-IN plasmid with XmaI-XhoI and ligating the RT-IN fragment with XmaI-XhoI cut pLR2P-vpr-p51-IRES-p66. The vpr-Δp51/p66 control plasmid was constructed to contain a translational stop codon at the first amino acid position of p51. Other derivatives of vpr-p51/p66 were constructed using PCR-based site-directed mutagenesis and cloning into the BglII-MluI or XmaI-XhoI sites of either p51 or p66, respectively. All clones were confirmed by nucleotide sequencing.

Transfection and Analysis of Virus Infectivity

DNA transfections were performed on monolayer cultures of 293T cells grown in 6-well plates using the calcium phosphate DNA precipitation method. Unless otherwise noted, each cell monolayer (well) was transfected with 6 μg of proviral DNA, 3 μg of the vpr-p51/p66 constructs and 1 μg of the vpr-lN construct. Culture supernatants from the 293T cells were collected 60 h post-transfection, clarified by low-speed centrifugation (1000×g, 10 min), and filtered through 0.45 μm pore-size sterile filers. The clarified supernatants were analyzed for HIV-1 p24 concentration by ELISA (Beckman-Coulter Inc.).

Virus infectivity was assessed using the TZM-b1 reporter cell line as described earlier (Wei et al. Antimicrob. Agents Chemother. 46:1895-905 (2002)). Briefly, virus containing supernatants were normalized for p24 antigen concentration, serially diluted (five-fold dilutions) and used to infect monolayer cultures of TZM-b1 cells. At 48 h post-infection, the cells were fixed and stained with 5-bromo-4-chloro-3-indolyl-β-D-galacto X-gal staining, the blue-stained cells were counted using a light microscope. Wells containing between 30 and 300 blue cells were used to calculate the infectious units of virus per ng of p24 antigen (IU/p24-ng).

The ability of trans-RT-containing virions to infect T cells was tested by quantitatively analyzing infection of the JLTRG-R5 reporter T cell line. 12-well flat-bottomed culture plates containing 1.0×10⁵ JLTRG-R5 cells were infected at a multipilicity of infection (MOI) of 5.0 (as determined by the TZM-b1 assay) for the wildtype Vpr-p51/p66 complemented virons. The other trans-RT-containing virion preparations were normalized for p24 antigen equivalent to that of that of the wildtype trans-RT. The total volume was adjusted to 1 ml and the infection was carried at 37° C. for 24 h. At 24 h post-infection, 1 ml of fresh RPMI 1640 was added to each well and culture was continued at 37° C. for an additional 48 h. Then the cells were washed (2×) in phosphate buffered saline (PBS). The cell pellet was resuspended in 50 μl PBS and then fixed in 1% paraformaldehyde (in PBS). The expression of EGFP was measured using a FACStar Plus flow cytometer with CellQuest software (Becton Dickinson).

Effects of NNRT is on Trans-RT Subunit Interaction

DNA transfections were performed on monolayer cultures of 293T cells grown in 6-well plates using the FuGENE 6 Transfection Reagent (Roche), as recommended by manufacturer. The M7 and vpr-p51/p66-based plasmids were used at a ratio of 2:1. At 24 h post-transfection, the specified concentrations of drug were added. Culture supernatants from the 293T cells were collected 60 h post-transfection, clarified by low-speed centrifugation (1000×g, 10 min), and filtered through 0.45 μm pore-size sterile filters. The clarified supernatants were processed for and analyzed by immunoblot as described below.

Immunobiot Analysis

Transfection-derived virions were concentrated by ultracentrifugation through 20% sucrose cushion (125,000×g, 2 hr, 4° C.) using a SW41 rotor (Beckman Inc.). Pellets were solubilized in Laemmli loading buffer (62.5 mM Tris-HCl [ph 6.8], 0.2% SDS, 5% 2-mercaptoethanol, 10% glycerol), boiled, and proteins were separated on 12.0% polyacrylamide gels containing SDS. Following electrophoresis, proteins were transferred to nitrocellulose (0.2-μm pore size) by electroblotting and incubated for 1 h at room temperature in blocking buffer (5% nonfat dry milk in PBS). The blocked blots were exposed to an appropriate primary antibody for 1 h in blocking buffer with constant mixing. After extension washing, bound antibodies were detected by chemiluminescence using horseradish peroxidase-conjugated species-specific secondary antibodies (Southern Biotechnology Associates, Inc.).

Inhibition of Trans-RT Using NRTI and NNRTI

Virions were derived by cotransfection of 293T cells with M7, vpr-p51/p66 and vpr-IN. The TZM-b1 cells were seeded overnight in 24-well plates at a concentration of 40,000 cells in 250 μl of medium per well. The culture medium was removed and replaced with 250 μl of DMEM containing 1% FBS and 2× drug concentrations (5-fold dilutions). 250 μl of virus suspension normalized for equal IU, as determined by TZM-b1 assay (diluted in DMEM containing 1% FBS and 80 μg/ml DEAE-dextran), was then added to the cells. The two RT drugs used in this analysis, 3TC and nevirapine, were at final concentrations ranging from 0.04-1.0 μM and 1.0-25.0 μM, respectively. The cells were fixed 48 h post-infection, stained with X-gal reagent, and the blue-stained cells were counted using a light microscope as described above. The 50% inhibition concentration (IC50) was measured with a 95% confidence interval.

### Example 6

### Trans-Complementation Analysis of Vpr-p51-IRES-p66

| | | | | | | p24 | Infectious | |
|---|---|---|---|---|---|---|---|---|
| | | | Blue | | Infectious | conc., | virions/ng | |
| | Viral | | Cells | Dilution | virions/ml | ng/ml | p24 | % of |
| # | DNA | trans-RT | A | B | A/B × 1000 = C | D | C/D | SG3 |
| 1 | pSG3- | pLR2P-vpr (cont) | 93 | 0.2 | 4.65E+05 | 714 | 6.51E+02 | 100.00 |
| | WT | | | | | | | |
| 2 | SG3-M7 | pLR2P-vpr (cont) | 0 | 5 | 0.00E+00 | 397 | 0.00E+00 | 0.00 |
| 3 | SG3- | pLR2P-vpr (cont) | 0 | 5 | 0.00E+00 | 688 | 0.00E+00 | 0.00 |
| | YMND | | | | | | | |
| 4 | SG3- | vpr-p51-IRES- | 66 | 1 | 6.60E+04 | 563 | 1.17E+02 | 18.00 |
| | YMND | p66 | | | | | | |
| 5 | SG3- | pLR2P-vpr (cont) | 0 | 5 | 0.00E+00 | 382 | 0.00E+00 | 0.00 |
| | YMAA | | | | | | | |
| 6 | SG3- | vpr-p51-IRES- | 21 | 5 | 4.20E+03 | 1072 | 3.92E+00 | 0.60 |
| | YMAA | p66 | | | | | | |

The table shows the trans-complementation analysis of Vpr-p51-IRES-p66 with viruses derived from proviral DNA containing mutations in the YMDD motif of RT, including YMAA and YMND. pLR2P-vpr is used as a negative control. The results show that the trans-heterodimeric RT can be expressed with proviral DNA, including mutant DNA, and incorporated into virions produced from the said cells, and function in reverse transcription. Methods: 293T cells were transfected with the indicated plasmid (either viral DNA or trans-RT DNA) DNAs. 48 hrs later the supernatant viruses were collected and analyzed for HIV-1 p24 antigen and infectivity using the JC53-BL reporter assay.

Throughout this application, various publications are referenced. The disclosures of these publications in their entireties are hereby incorporated by reference into this application in order to more fully describe the state of the art to which this invention pertains.

### References

Amacker, M., Hottiger, M. and Hubscher, U. (1995) Feline immunodeficiency virus reverse transcriptase: expression, functional characterization, and reconstitution of the 66- and 51-kilodalton subunits. J. Virol., 69, 6273-6279.
Arnold, E., Jacobo-Molina, A., Nanni, R. G., Williams, R. L., Lu, X., Ding, J., Clark, A. D., Jr., Zhang, A., Ferris, A. L., Clark, P. and et al. (1992) Structure of HIV-1 reverse transcriptase/DNA complex at 7 A resolution showing active site locations. Nature, 357, 85-89.
Arts, E. J., Li, X., Gu, Z., Kleiman, L., Parniak, M. A. and Wainberg, M. A. (1994) Comparison of deoxyoligonucleotide and tRNA(Lys-3) as primers in an endogenous human immunodeficiency virus-1 in vitro reverse transcription/template-switching reaction. J. Biol. Chem., 269, 14672-14680.
Baillon, J. G., Nashed, N. T., Kumar, A., Wilson, S. H. and Jerina, D. M. (1991) A leucine zipper-like motif may mediate HIV reverse transcriptase subunit binding. New Biol., 3, 1015-1019.
Balzarini, J. “Current status of the non-nucleoside reverse transcriptase inhibitors of human immunodeficiency virus type 1.” Curr. Top. Meal Chem. 4:921-44 (2004).
Boyer, P. L., Ding, J., Arnold, E. and Hughes, S. H. (1994) Subunit specificity of mutations that confer resistance to nonnucleoside inhibitors in human immunodeficiency virus type 1 reverse transcriptase. Antimicrob. Agents Chemother., 38, 1909-1914.
Boyer, P. L., Ferris, A. L. and Hughes, S. H. (1992) Cassette mutagenesis of the reverse transcriptase of human immunodeficiency virus type 1. J. Virol., 66, 1031-1039.
Cabodevilla, J. F., Odriozola, L., Santiago, E. & Martinez-Irujo, J. J. “Factors affecting the dimerization of the p66 form of HIV-1 reverse transcriptase.” Eur. J. Biochem. 268, 1163-72 (2001).
di Marzo Veronese, F., Copeland, T. D., DeVico, A. L., Rahman, R., Oroszlan, S., Gallo, R. C. and Sarngadharan, M. G. (1986) Characterization of highly immunogenic p66/p51 as the reverse transcriptase of HTLV-III/LAV. Science, 231, 1289-1291.
Ding, J., Das, K., Hsiou, Y., Sarafianos, S. G., Clark, A. D., Jr., Jacobo-Molina, A., Tantillo, C., Hughes, S. H. and Arnold, E. (1998) Structure and functional implications of the polymerase active site region in a complex of HIV-1 RT with a double-stranded DNA template-primer and an antibody Fab fragment at 2.8 A resolution. J. Mol. Biol., 284, 1095-1111.
Divita, G., Rittinger, K., Geourjon, C., Deleage, G. & Goody, R. S. “Dimerization kinetics of HIV-1 and HIV-2 reverse transcriptase: a two step process. J. Mol. Biol. 245:508-21 (1995)
Divita, G., Baillon, J. G., Rittinger, K., Chermann, J. C. & Goody, R. S. “Interface peptides as structure-based human immunodeficiency virus reverse transcriptase inhibitors. J. Biol. Chem. 270:28642-6 (1995)
Divita, G., Rittinger, K., Restle, T., Immerdorfer, U. & Goody, R. S. Conformational stability of dimeric HIV-1 and HIV-2 reverse transcriptases.” Biochemistry 34:16337-46 (1995).
Divita, G., Restle, T., Goody, R. S., Chermann, J. C. and Baillon, J. G. (1994) Inhibition of human immunodeficiency virus type 1 reverse transcriptase dimerization using synthetic peptides derived from the connection domain. J. Biol. Chem., 269, 13080-13083.
Dubay, J. W., Roberts, S. J., Hahn, B. H. and Hunter, E. (1992) Truncation of the human immunodeficiency virus type 1 transmembrane glycoprotein cytoplasmic domain blocks virus infectivity. J. Virol., 66, 6616-6625.
Duke, G. M., Hoffman, M. A. and Palmenberg, A. C. (1992) Sequence and structural elements that contribute to efficient encephalomyocarditis virus RNA translation. J. Viral., 66, 1602-1609.
Fassati, A. and Goff, S. P. (2001) Characterization of intracellular reverse transcription complexes of human immunodeficiency virus type 1. J. Virol., 75, 3626-3635.
Freed, E. O. and Martin, M. A. (2001) HIV and Their Replication. In Knipe, D. M., Howley, P. M., Griffin, D. E., Lamb, R. A., Martin, M. A., Roizman, B. and Straus, S. E. (eds.), Fields Virology. Lippincott Williams & Wilkins, Philadelphia, Pa., Vol. 2, pp. 1971-2042.
Ghosh, M., Jacques, P. S., Rodgers, D. W., Ottman, M., Darlix, J. L. and Le Grice, S. F. (1996) Alterations to the primer grip of p66 HIV-1 reverse transcriptase and their consequences for template-primer utilization. Biochemistry, 35, 8553-8562.
Ghosh, S. K., Fultz, P. N., Keddie, E., Saag, M. S., Sharp, P. M., Hahn, B. H. and Shaw, G. M. (1993) A molecular clone of HIV-1 tropic and cytopathic for human and chimpanzee lymphocytes. Virology, 194, 858-864.
Goel, R., Beard, W. A., Kumar, A., Casas-Finet, J. R., Strub, M. P., Stahl, S. J., Lewis, M. S., Bebenek, K., Becerra, S. P., Kunkel, T. A. & et al. “Structure/function studies of HIV-1(1) reverse transcriptase: dimerization-defective mutant L289K. Biochemistry 32:13012-8 (1993).
Harris, D., Yadav, P. N. and Pandey, V. N. (1998) Loss of polymerase activity due to Tyr to Phe substitution in the YMDD motif of human immunodeficiency virus type-1 reverse transcriptase is compensated by Met to Val substitution within the same motif. Biochemistry, 37, 9630-9640.
Hizi, A., McGill, C. and Hughes, S. H. (1988) Expression of soluble, enzymatically active, human immunodeficiency virus reverse transcriptase in Escherichia coli and analysis of mutants. Proc. Natl. Acad. Sci. USA, 85, 1218-1222.
Hostomsky, Z., Hostomska, Z., Fu, T. B. and Taylor, J. (1992) Reverse transcriptase of human immunodeficiency virus type 1: functionality of subunits of the heterodimer in DNA synthesis. J. Virol., 66, 3179-3182.
Hottiger, M., Podust, V. N., Thimmig, R. L., McHenry, C. and Hubscher, U. (1994) Strand displacement activity of the human immunodeficiency virus type 1 reverse transcriptase heterodimer and its individual subunits. J Biol. Chem., 269, 986-991.
Huang, H., Chopra, R., Verdine, G. L. and Harrison, S. C. (1998) Structure of a covalently trapped catalytic complex of HIV-1 reverse transcriptase: implications for drug resistance. Science, 282, 1669-1675.
Hughes, S. H. (2001) Molecular matchmaking: NNRTIs can enhance the dimerization of HIV type 1 reverse transcriptase. Proc. Natl. Acad. Sci. USA, 98, 6991-6992.
Jacques, P. S., Wohrl, B. M., Howard, K. J. and Le Grice, S. F. (1994) Modulation of HIV-1 reverse transcriptase function in “selectively deleted” p66/p51 heterodimers. J. Biol. Chem., 269, 1388-1393.
Jonckheere, H., Taymans, J. M., Balzarini, J., Velazquez, S., Camarasa, M. J., Desmyter, J., De Clercq, E. and Anne, 3. (1994) Resistance of HIV-1 reverse transcriptase against [2′,5′-bis-O-(tert-butyldimethylsilyl)-3′-spiro-5″-(4″-amino-1″,2″-oxathiole-2″,2″-dioxide)] (TSAO) derivatives is determined by the mutation Glu138→Lys on the p51 subunit. J. Biol. Chem., 269, 25255-25258.
Kamer, G. and Argos, P. (1984) Primary structural comparison of RNA-dependent polymerases from plant, animal and bacterial viruses. Nucleic Acids Res., 12, 7269-7282.
Kimpton, J. and Emerman, M. (1992) Detection of replication-competent and pseudotyped human immunodeficiency virus with a sensitive cell line on the basis of activation of an integrated beta-galactosidase gene. J. Virol., 66, 2232-2239.
Kohlstaedt, L. A., Wang, J., Friedman, J. M., Rice, P. A. and Steitz, T. A. (1992) Crystal structure at 3.5 A resolution of HIV-1 reverse transcriptase complexed with an inhibitor. Science, 256, 1783-1790.
Kozak, M. (1987) At least six nucleotides preceding the AUG initiator codon enhance translation in mammalian cells. J. Mol. Biol., 196, 947-950.
Kutsch, O., Levy, D. N., Bates, P. J., Decker, J., Kosloff, B. R., Shaw, G. M., Priebe, W. & Benveniste, E. N. “Bis-anthracycline antibiotics inhibit human immunodeficiency virus type 1 transcription. Antimicrob. Agents Chemother. 48:1652-63 (2004).
Larder, B., Purifoy, D., Powell, K. and Darby, G. (1987a) AIDS virus reverse transcriptase defined by high level expression in Escherichia coli. EMBO J., 6, 3133-3137.
Larder, B. A., Purifoy, D. J., Powell, K. L. and Darby, G. (1987b) Site-specific mutagenesis of AIDS virus reverse transcriptase. Nature, 327, 716-717.
Le Grice, S. F., Naas, T., Wohlgensinger, B. and Schatz, O. (1991) Subunit-selective mutagenesis indicates minimal polymerase activity in heterodimer-associated p51 HIV-1 reverse transcriptase. EMBO J., 10, 3905-3911.
Lightfoote, M. M., Coligan, J. E., Folks, T. M., Fauci, A. S., Martin, M. A. and Venkatesan, S. (1986) Structural characterization of reverse transcriptase and endonuclease polypeptides of the acquired immunodeficiency syndrome retrovirus. J. Virol., 60, 771-775.
Liu, H., Wu, X., Xiao, H., Conway, J. A. and Kappes, J. C. (1997) Incorporation of functional human immunodeficiency virus type 1 integrase into virions independent of the Gag-Pol precursor protein. J. Virol., 71, 7704-7710.
Lowe, D. M., Parmar, V., Kemp, S. D. and Larder, B. A. (1991) Mutational analysis of two conserved sequence motifs in HIV-1 reverse transcriptase. FEBS Lett., 282, 231-234.
Loya, S., Gao, H. Q., Avidan, O., Boyer, P. L., Hughes, S. H. and Hizi, A. (1997) Subunit-specific mutagenesis of the cysteine 280 residue of the reverse transcriptase of human immunodeficiency virus type 1: effects on sensitivity to a specific inhibitor of the RNase H activity. J. Virol., 71, 5668-5672.
Lu, Y. L., Spearman, P. and Ratner, L. (1993) Human immunodeficiency virus type 1 viral protein R localization in infected cells and virions. J. Virol., 67, 6542-6550.
Madrid, M., Lukin, J. A., Madura, J. D., Ding, J. & Arnold, E. “Molecular dynamics of HIV-1 reverse transcriptase indicates increased flexibility upon DNA binding.” Proteins 45:176-82 (2001).
Menendez-Arias, L., Abraha, A., Quinones-Mateu, M. E., Mas, A., Camarasa, M. J. and Arts, E. J. (2001) Functional characterization of chimeric reverse transcriptases with polypeptide subunits of highly divergent HIV-1 group M and O strains. J. Biol. Chem., 276, 27470-27479.
Morris, M. C., Robert-Hebmann, V., Chaloin, L., Mery, J., Heitz, F., Devaux, C., Goody, R. S. and Divita, G. (1999) A new potent HIV-1 reverse transcriptase inhibitor. A synthetic peptide derived from the interface subunit domains. J. Biol. Chem., 274, 24941-24946.
Morris, M. D., Berducou, C., Mery, J., Heitz, F. & Divita, G. “The thumb domain of the P51-subunit is essential for activation of HIV reverse transcriptase. Biochemistry 38:15097-103 (1999).
Mulky, A., Sarafianos, S. G., Arnold, E., Wu, X. & Kappes, J. C. “Subunit-specific analysis of the human immunodeficiency virus type 1 reverse transcriptase in vivo.” J. Virol. 78:7089-96 (2004).
Ochsenbauer-Jambor, C., Jones, J., Zammit, K. & Kutsch, O. “Optimized HIV-1 drug screening in a 384-well plate format using EGFP as a read-out, submitted (2004).
Olivares, I., Gutierrez-Rivas, M., Lopez-Galindez, C. & Menendez-Arias, L. “HIV-1 reverse transcriptase: critical role of Phe-130 for p51 function and second-site revertant restoring viral replication capacity.” Virology 324, 400-11 (2004).
Pandey, P. K., Kaushik, N., Talele, T. T., Yadav, P. N. & Pandey, V. N. “The beta7-beta8 loop of the p51 subunit in the heterodimeric (p66/p51) human immunodeficiency virus type 1 reverse transcriptase is essential for the catalytic function of the p66 subunit.” Biochemistry 40, 9505-12 (2001).
Paxton, W., Connor, R. I. and Landau, N. R. (1993) Incorporation of Vpr into human immunodeficiency virus type 1 virions: requirement for the p6 region of gag and mutational analysis. J. Virol., 67, 7229-7237.
Platt, E. J., Wehrly, K., Kuhmann, S. E., Chesebro, B. and Kabat, D. (1998) Effects of CCR5 and CD4 cell surface concentrations on infections by macrophagetropic isolates of human immunodeficiency virus type 1. J. Virol., 72, 2855-2864.
Prasad, V. R. and Goff, S. P. (1989) Linker insertion mutagenesis of the human immunodeficiency virus reverse transcriptase expressed in bacteria: definition of the minimal polymerase domain. Proc. Natl. Acad. Sci. USA, 86, 3104-3109.
Quillent, C., Borman, A. M., Paulous, S., Dauguet, C. & Clavel, F. “Extensive processing and particle maturation.” Virology 219:29-36 (1996).
Restle, T., Muller, B. and Goody, R. S. (1990) Dimerization of human immunodeficiency virus type 1 reverse transcriptase. A target for chemotherapeutic intervention. J. Biol. Chem., 265, 8986-8988.
Rodriguez-Barrios, F., Perez, C., Lobaton, E., Velazquez, S., Chamorro, C., San-Felix, A., Perez-Perez, M. J., Camarasa, M. J., Pelemans, H., Balzarini, J. & Gago, F. “Identification of a putative binding site for [2′,5′-bis-O-(tert-butyldimethylsilyl)-beta-D-ribofuranosyl]-3′-spiro-5″-(4″-amino-1″,2″-oxathiole-2″,2″-eioxide)thymine (TSAO) derivatives at the p51-p66 interface of HIV-1 reverse transcriptase.” J. Meal Chem. 44: 1853-65 (2001).
Sarafianos, S. G., Das, K., Tantillo, C., Clark, A. D., Jr., Ding, J., Whitcomb, J. M., Boyer, P. L., Hughes, S. H. and Arnold, E. (2001) Crystal structure of HIV-1 reverse transcriptase in complex with a polypurine tract RNA:DNA. EMBO J., 20, 1449-1461.
Sarafianos, S. G., Das, K., Ding, J., Boyer, P. L., Hughes, S. H. & Arnold, E. “Touching the heart of HIV-1 drug resistance: the fingers close down on the dNTP at the polymerase active site.” Chem. Biol. 6:R137-46 (1999).
Sevilya, Z., Loya, S., Hughes, S. H. & Hizi, A. “The ribonuclease H activity of the reverse transcriptases of human immunodeficiency viruses type 1 and type 2 is affected by the thumb subdomain of the small protein subunits. J. Mol. Biol. 311:957-71 (2001).
Shehu-Xhilaga, -M., Hill, M., Marshall, J. A., Kappes, J., Crowe, S. M. and Mak, J. (2002) The conformation of the mature dimeric human immunodeficiency virus type 1 RNA genome requires packaging of pol protein. J. Virol., 76, 4331-4340.
Sluis-Cremer, N., Arion, D. & Parniak, M. A. “Destabiiization of the HIV-1 reverse transcriptase dimer upon interaction with N-acyl hydrazone inhibitors. “Mol Pharmacol 62:398-405 (2002).
Sluis-Cremer, N., Dmitrienko, G. I., Balzarini, J., Camarasa, M. J. and Parniak, M. A. (2000) Human immunodeficiency virus type 1 reverse transcriptase dimer destabilization by 1-[Spiro[4″-amino-2″,2″-dioxo-1″,2″-oxathiole-5″,3′-[2′,5′-bis-O-(tert-butyldimethylsilyl)-beta-D-ribofuranosyl]]]-3-ethylthy mine. Biochemistry, 39, 1427-1433.
Smith, D. B. and Johnson, K. S. (1988) Single-step purification of polypeptides expressed in Escherichia coli as fusions with glutathione S-transferase. Gene, 67, 31-40.
Tachedjian, G., Moore, K. D., Goff, S. P. & Sluis-Cremer, N. “Efavirenz enhances the proteolytic processing a an HIV-1 pol polyprotein precursor and reverse transcriptase homodimer formation. FEBS Lett 579:379-384 (2005).
Tachedjian, G., Aronson, H. E., de los Santos, M., Seehra, J., McCoy, J. M. and Goff, S. P. (2003) Role of residues in the tryptophan repeat motif for HIV-1 reverse transcriptase dimerization. J. Mol. Biol., 326, 381-396.
Tachedjian, G., Orlova, M., Sarafianos, S. G., Arnold, E. & Golf, S. P. “Nonnucleoside reverse transcriptase inhibitors are chemical enhancers of dimerization of the HIV type 1 reverse transcriptase.” Proc. Natl. Acad. Sci. USA 98: 7188-93 (2001).
Telesnitsky, A. and Goff, S. P. (1997) Reverse Transcriptase and the Generation of Retroviral DNA. In Coffin, J. M., Hughes, S. H. and Varmus, H. E. (eds.), Retroviruses. Cold Spring Harbor Laboratory Press, Cold Spring Harbor, N.Y., pp. 121-160.
Temiz, N. A. & Bahar, I. “Inhibitor binding alters the directions of domain motions in HIV-1 reverse transcriptase.” Proteins 49:61-70 (2002).
Toh, H., Hayashida, H. and Miyata, T. (1983) Sequence homology between retroviral reverse transcriptase and putative polymerases of hepatitis B virus and cauliflower mosaic virus. Nature, 305, 827-829.
Tomonaga, K., Itagaki, S. I., Kashiwase, H., Kawaguchi, Y., Inoshima, Y., Ikeda, Y. & Mikami, T. “Characterization of an integrase mutant of feline immunodeficiency virus. Arch. Virol. 143, 1-14 (1998).
Tuske, S., Sarafianos, S. C., Clark, A. D., Jr., Ding, J., Naeger, L. K., White, K. L., Miller, M. D., Gibbs, C. S., Boyer, P. L., Clark, P., Wang, G., Gaffney, B. L., Jones R. A., Jerina, D. M., Hughes, S. H. & Arnold, E. “Structures of HIV-1 RT-DNA complexes before and after incorporation of the anti-AIDS drug tenofovir.” Nat. Struct. Mol. Biol. 11:469-74 (2004).
Wakefield, J. K., Jablonski, S. A. and Morrow, C. D. (1992) In vitro enzymatic activity of human immunodeficiency virus type 1 reverse transcriptase mutants in the highly conserved YMDD amino acid motif correlates with the infectious potential of the proviral genome. J. Virol., 66, 6806-6812.
Wei, X., Decker, J. M., Liu, H., Zhang, Z., Arani, R. B., Kilby, J. M., Saag, M. S., Wu, X., Shaw, G. M. and Kappes, J. C. (2002) Emergence of resistant human immunodeficiency virus type 1 in patients receiving fusion inhibitor (T-20) monotherapy. Antimicrob. Agents Chemother., 46, 1895-1905.
Wu, X., Conway, J. A., Kim, J. and Kappes, J. C. (1994) Localization of the Vpx packaging signal within the C terminus of the human immunodeficiency virus type 2 Gag precursor protein. J. Virol., 68, 6161-6169.
Wu, X., Liu, H., Xiao, H., Conway, J. A., Hehl, E., Kalpana, G. V., Prasad, V. and Kappes, J. C. (1999) Human immunodeficiency virus type 1 integrase protein promotes reverse transcription through specific interactions with the nucleoprotein reverse transcription complex. J. Virol., 73, 2126-2135.
Wu, X., Liu, H., Xiao, H., Conway, J. A., Hunter, E. and Kappes, J. C. (1997) Functional RT and IN incorporated into HIV-1 particles independently of the Gag/Pol precursor protein. EMBO J., 16, 5113-5122.
Wu, X., Liu, H., Xiao, H., Kim, J., Seshaiah, P., Natsoulis, G., Boeke, J. D., Hahn, B. H. and Kappes, J. C. (1995) Targeting foreign proteins to human immunodeficiency virus particles via fusion with Vpr and Vpx. J. Virol., 69, 3389-3398.
Yu, Q., Ottmann, M., Pechoux, C., Le Grice, S. & Dariix, J.-L. “Mutations in the Primer Grip of Human Immunodeficiency Virus Type 1 Reverse Transcriptase Impair Proviral DNA Synthesis and Virion Maturation.” Virology 219:29-36 (1996).
Zack, J. A., Arrigo, S. J., Weitsman, S. R., Go, A. S., Haislip, A. and Chen, I. S. (1990) HIV-1 entry into quiescent primary lymphocytes: molecular analysis reveals a labile, latent viral structure. Cell, 61, 213-222.

What is claimed is:

1. A cell comprising: (i) a vector comprising a p66 subunit, a p51 subunit, and Vpr, wherein Vpr and p51 are expressed as a fusion protein; (ii) and a retrovirus proviral DNA.

2. A method of screening for a compound that inhibits viral reverse transcriptase comprising: a) contacting the cell of claim 1 with the compound, and b) comparing the level of viral infectivity in the presence of the compound with the level of viral infectivity in the absence of the compound, wherein a decreased level of infectivity in the presence of the compound indicates that the compound inhibits reverse transcriptase.

3. A method of screening for a compound that inhibits viral reverse transcriptase comprising: a) contacting the cell of claim 1 with a compound; and b) comparing the level of p66 in virus particles generated by the cell.

4. A method of screening for a compound that inhibits viral reverse transcriptase comprising: a) contacting the cell of claim 1 with a compound; and b) comparing the level of reverse transcriptase in virus particles generated by the cell.

5. The method of any one of claims 2-4, wherein the virus is a lentivirus.

6. The method of claim 5, wherein the virus is HIV-1.

7. The method of any one of claims 2-4, wherein the p51 and p66 subunits are expressed in trans in the cell.

8. The method of any one of claims 2-4, wherein the p51 and p66 subunits are expressed on different messenger RNAs.

9. The method of any one of claims 2-4, wherein the p51 and p66 subunits are expressed on the same messenger RNAs.

10. The method of any one of claims 2-4, wherein expression of Vpr-p51 incorporates p66 protein into viral particles.

11. The method of any one of claims 1-4, wherein p51 interacts with p66 protein.

12. The method of any one of claims 1-4, wherein p51 contains a mutation, insertion, or deletion.

13. The method of any one of claims 1-4, wherein p66 contains a mutation, insertion, or deletion.

14. The method of any one of claims 1-4, wherein the plasmid also expresses an internal ribosome entry site (IRES).

15. A method of screening for a compound that affects dimerization of a p66 subunit polypeptide of reverse transcriptase and a p51 subunit polypeptide of reverse transcriptase comprising: a) contacting the cell of claim 1 with the compound, and b) comparing the level of complex formation in the presence of the compound with the level of complex formation in the absence of the compound, a change in the level of complex formation indicating that the compound affects dimerization of the p66 subunit and a p51 subunit.

16. A method of screening for a compound that inhibits dimerization of a p66 subunit polypeptide of reverse transcriptase and a p51 subunit polypeptide of reverse transcriptase comprising: a) contacting the cell of claim 1 with the compound, and b) comparing the level of complex formation in the presence of the compound with the level of complex formation in the absence of the compound, a lower level of complex formation indicating that the compound inhibits dimerization of the p66 subunit and a p51 subunit.

17. A method of screening for a compound that enhances dimerization of a p66 subunit polypeptide of reverse transcriptase and a p51 subunit polypeptide of reverse transcriptase comprising: a) contacting the cell of claim 1 with the compound, and b) comparing the level of complex formation in the presence of the compound with the level of complex formation in the absence of the compound, a higher level of complex formation indicating that the compound enhances dimerization of the p66 subunit and a p51 subunit.

18. A method of making a pharmaceutical composition which comprises: a) determining whether a compound inhibits reverse transcriptase by the method of claim 2; and b) admixing the compound with a pharmaceutically acceptable carrier.

19. A method of inhibiting viral reverse transcriptase comprising contacting (1) the p51 subunit polypeptide, (2) the p66 subunit polypeptide, or (3) both the p51 subunit polypeptide and the p66 subunit polypeptide, with an effective amount of the compound identified by the method of claim 2, thereby inhibiting viral reverse transcriptase.

20. A method of inhibiting dimerization of a p51 subunit polypeptide of HIV-1 reverse transcriptase and a p66 subunit polypeptide of HIV-1 reverse transcriptase, which comprises contacting either (1) the p51 subunit polypeptide, (2) the p66 subunit polypeptide, or (3) both the p51 subunit polypeptide and the p66 subunit polypeptide, with an effective amount of the compound identified by the method of claim 16, thereby inhibiting dimerization of the p51 subunit polypeptide of HIV-1 reverse transcriptase and a p66 subunit polypeptide of HIV-1 reverse transcriptase.

21. A method of enhancing dimerization of a p51 subunit polypeptide of HIV-1 reverse transcriptase and a p66 subunit polypeptide of HIV-1 reverse transcriptase, which comprises contacting either (1) the p51 subunit polypeptide, (2) the p66 subunit polypeptide, or (3) both the p51 subunit polypeptide and the p66 subunit polypeptide, with an effective amount of the compound identified by the method of claim 17, thereby enhancing dimerization of the p51 subunit polypeptide of HIV-1 reverse transcriptase and a p66 subunit polypeptide of HIV-1 reverse transcriptase.

22. The method of claim 21, wherein the HIV-1 reverse transcriptase is present in a subject.

23. The method of claim 22, wherein the compound is administered to the subject orally, intravenously, subcutaneously, intramuscularly, topically or by liposome-mediated delivery.

24. A compound identified by the method of claim 2.

25. A compound identified by the method of claim 15.

26. A compound identified by the method of claim 16.

27. A composition which comprises the compound of claim 24 and a carrier.

28. The compound of claim 24, wherein the compound is capable of inhibiting HIV-1.

29. The compound of claim 28, wherein the compound is a nonnucleoside reverse transcriptase inhibitor.

30. An expression cassette comprising LTR-vpr-p51-IRES-p66.

31. The expression cassette of claim 30, wherein the nucleic acid comprises SEQ ID NO: 1.

32. The method of claim 2, wherein the HIV or SIV particles are derived by genes expressed in the cell and wherein the genes contain one or more nucleotide mutations.

33. A transgenic animal expressing vpr-p51/66.

34. A cell line comprising an exogenous nucleic acid, the nucleic acid comprising vpr-p51/66.

35. The cell line of claim 34, wherein the cell expresses viral nucleic acids.

36. The cell line of claim 34, wherein the cell can be induced to express viral nucleic acids by contacting the cell with a stimulus.


SEQUENCE LISTING (21 sequences)

SEQ ID NO: 1 - DNA, 858 bp, Artificial Sequence
  Description of Artificial Sequence; note = synthetic construct
gtttaaacgccaccatggagcaggcccccgaggaccagggcccccagagggagccccacaacgagtggaccctggagctgctggaggagctgaagagggaggccgtgaggcacttccccaggccctggctgcacggcctgggccagcacatctacgagacctacggcgacacctgggccggcgtggaggccatcatcaggatcctgcagcagctgctgttcatccacttcaggatcggctgccagcacagcaggatcggcatcatccagcagaggagggccaggaggaacggcgccagcaggagctagtttaaacactgcacagagagacaggctaattttttagggaaaatttggccttccaacaaagggaggccagggaattttctccagaacaggccagagccaacagccccacccgcagagagcctcgggttcggagaggagatagccccctccccgaaacaagagccgaaggaaaaggagttataccccttaacctccctcaaatcactctttggcagcgacccctagtcacagtaagaatagggggacagctaatagaagccctgttagacacaggagcagatgatacagtgttagaagatataaatttaccaggaaaatggaaaccaaaaatgatagggggaattggtggtcttatcaaagtaagacagtatgatcaaatacttatagaaatttgtggaaaaaaggctatagggacagtattagtaggacctacacctatcaacataattgggagaaatatgttgactcagattggttgtactttaaattttccaattagtcctattgaaactgtaccagtaaaattaaagccaggaatggatggtccaaa

SEQ ID NO: 2 - protein, 96 aa, Artificial Sequence
  Description of Artificial Sequence; note = synthetic construct
MEQAPEDQGPPREPYNAWTLELLEELKSEAVRHFPRVWLHGLGQHIYETYGDTWAGVEAIIRILQQLLFIHFRIGCQHSRIGITRQRRARNGASRS

SEQ ID NO: 3 - DNA, 315 bp, Artificial Sequence
  Description of Artificial Sequence; note = synthetic construct
gtttaaacgccaccatggagcaggcccccgaggaccagggcccccagagggagccccacaacgagtggaccctggagctgctggaggagctgaagagggaggccgtgaggcacttccccaggccctggctgcacggcctgggccagcacatctacgagacctacggcgacacctgggccggcgtggaggccatcatcaggatcctgcagcagctgctgttcatccacttcaggatcggctgccagcacagcaggatcggcatcatccagcagaggagggccaggaggaacggcgccagcaggagctagtttaaac

SEQ ID NO: 4 - protein, 440 aa, Artificial Sequence
  Description of Artificial Sequence; note = synthetic construct
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETF

SEQ ID NO: 5 - protein, 440 aa, Artificial Sequence; identical to SEQ ID NO: 4
  Description of Artificial Sequence; note = synthetic construct

SEQ ID NO: 6 - protein, 170 aa, Artificial Sequence
  Description of Artificial Sequence; note = synthetic construct
KEGHQMKECTERQANFLGKIWPSHKGRPGNFLQSRPEPTAPPEESFRCGEEKTTPPQKPEQTDKELYPLASLRSLFGQRPLVTIKIGGQLKEALLDTGADDTVLEDMSLPGKWKPKMIGGIGGFIKVRQYDQIPIEICGHKAIGTVLIGPTPVNIIGRNLLTQIGCTLNF

SEQ ID NO: 7 - DNA, 511 bp, Artificial Sequence
  Description of Artificial Sequence; note = synthetic construct
aaaggaaggacaccaaatgaaagaatgcactgagagacaggctaattttttagggaaaatctggccttcccacaagggaaggccagggaactttctccagagcagaccagagccaacagccccaccagaagagagcttcaggtgtggggaggagaaaacaactccccctcagaagccggagcagacagacaaggaactgtatcctttagcttccctcagatcactctttggcaacgacccctcgtcacaataaagataggggggcagctaaaggaagctctattagatacaggagcagatgatacagtattagaagacatgagtttgccaggaaaatggaagccaaaaatgatagggggaattggaggttttatcaaagtaagacagtatgatcagatacctatagaaatctgtgggcataaagctataggtacagtattaataggaccaacacctgtcaacataattggaagaaatctgttgacacagattggttgcactttaaattttc

SEQ ID NO: 8 - protein, 4 aa, Artificial Sequence
  Description of Artificial Sequence; note = synthetic construct
YMDD

SEQ ID NO: 9 - protein, 4 aa, Artificial Sequence
  Description of Artificial Sequence; note = synthetic construct
YXDD

SEQ ID NO: 10 - DNA, 26 bp, Artificial Sequence
  Description of Artificial Sequence; note = synthetic construct
aagcccgggatggatggcccaaaagt

SEQ ID NO: 11 - DNA, 45 bp, Artificial Sequence
  Description of Artificial Sequence; note = synthetic construct
tcctaaacgcgtctccctctaagctgctcaatttacttagaaagt

SEQ ID NO: 12 - DNA, 45 bp, Artificial Sequence
  Description of Artificial Sequence; note = synthetic construct
actttctaagtaaattgagcagcttagagggagacgcgtttagga

SEQ ID NO: 13 - DNA, 25 bp, Artificial Sequence
  Description of Artificial Sequence; note = synthetic construct
tatgtcgacacccaattatgaaaag

SEQ ID NO: 14 - DNA, 32 bp, Artificial Sequence
  Description of Artificial Sequence; note = synthetic construct
tagatcagatctgttgactcagattggttgca

SEQ ID NO: 15 - DNA, 32 bp, Artificial Sequence
  Description of Artificial Sequence; note = synthetic construct
atctacacgcgtttagaaggtttctgcgcctt

SEQ ID NO: 16 - DNA, 32 bp, Artificial Sequence
  Description of Artificial Sequence; note = synthetic construct
ttattaacgcgtccgcccctctccctcccccc

SEQ ID NO: 17 - DNA, 69 bp, Artificial Sequence
  Description of Artificial Sequence; note = synthetic construct
ccatcccgggctttaattttactggtacagtttcaataggactaatgggtcccatggtattatcgtctt

SEQ ID NO: 18 - DNA, 21 bp, Artificial Sequence
  Description of Artificial Sequence; note = synthetic construct
agcttgccttgagtgcttcaa

SEQ ID NO: 19 - DNA, 26 bp, Artificial Sequence
  Description of Artificial Sequence; note = synthetic construct
ctgctagagattttccacactgacta

SEQ ID NO: 20 - DNA, 21 bp, Artificial Sequence
  Description of Artificial Sequence; note = synthetic construct
ggctagctagggaacccactg

SEQ ID NO: 21 - DNA, 22 bp, Artificial Sequence
  Description of Artificial Sequence; note = synthetic construct
atactgacgctctcgcacccat